(19) United States
(12) Patent Application Publication
(10) Pub. No.: US 2026/0146997 A1
(43) Pub. Date: May 28, 2026

(54) Activation-dependent immune dysfunction traits enable identification of prodromal Parkinson's

(71) Applicant: UNIVERSITY OF FLORIDA RESEARCH FOUNDATION, INCORPORATED, Gainesville, FL (US)

(72) Inventors: Malú Mariadelourdes Gámez Tansey, Gainesville, FL (US); Rebecca Wallings, Gainesville, FL (US); Julian Mark, Gainesville, FL (US)

(21) Appl. No.: 19/399,098

(22) Filed: Nov. 24, 2025

Related U.S. Application Data
(60) Provisional application No. 63/723,821, filed on Nov. 22, 2024.

(51) Int. Cl. G01N 33/569; G01N 15/01; G01N 15/10; G01N 15/1434; G01N 33/58; G01N 33/68

(52) U.S. Cl. CPC G01N 33/56972; G01N 15/01; G01N 15/1434; G01N 33/582; G01N 33/6896; G01N 2015/1006; G01N 2333/525; G01N 2333/5421; G01N 2800/2835; G01N 2800/50; G01N 2800/52

(57) ABSTRACT

Idiopathic Parkinson's disease (iPD) is a multi-system disorder, and the debilitating motor stage of iPD can be preceded for years by a prodromal stage characterized by non-motor symptoms like REM sleep behavior disorder (RBD) and gastrointestinal symptoms. Widespread immune dysregulation has been reported in clinically diagnosed iPD, but the existence of immune deficits during the prodromal stage has yet to be thoroughly investigated. Here, it was shown that multiple stages of iPD, including the pre-motor prodromal stage, can be stratified according to the immuno-metabolic response to stimulation of peripheral blood immune cells ex vivo. Peripheral blood monocytes from RBD patients displayed increased stimulation-dependent secretion of inflammatory cytokines, including TNF, IL-1β, and IL-8, which peaks in the prodromal stage and successively diminishes as PD progresses. Furthermore, T lymphocyte mitochondrial health was correlated with stimulation-evoked cytokine secretion across patients with RBD, early-stage iPD, and moderate-stage iPD. The results disclosed here have broad implications for mechanistic understanding of how peripheral inflammation may drive disease progression, and it reveals novel biomarkers to enable patient stratification and progression monitoring for clinical trials based on immune endophenotypes.

### FUNDING SUPPORT

This invention was created in whole or in part by Grant Number MJFF-023925, received by The Michael J. Fox Foundation for Parkinson's Research and Shake It Up Australia Foundation; and Grant Number 18891, received by The Michael J. Fox Foundation for Parkinson's Research.

### BACKGROUND

Parkinson's disease (PD) is a multi-system neurodegenerative disease for which there are no effective disease modifying therapies. Neuroprotective strategies have been largely ineffective because the majority of dopaminergic neurons in the substantia nigra pars compacta (SNpc) have already been lost by the time motor symptoms present and clinical diagnosis can be made¹. This underscores the need for accessible biomarkers that facilitate early diagnosis and identify patient endophenotypes for superior recruitment and assignment into clinical trials.

### SUMMARY

Significant attention has been directed towards patients with isolated REM sleep behavior disorder (RBD), as this disorder is a strong predictive marker of pre-motor prodromal PD^{2,3}. RBD is characterized by loss of muscle atonia during REM sleep and the physical acting out of dreams that are often intense or violent⁴. Approximately 80% of RBD patients will develop a neurodegenerative disease within 10.5 years of RBD diagnosis, and the plurality (43%) of those who convert will develop PD². Thus, studying individuals with RBD could reveal novel biomarkers for earlier diagnosis of PD and grant insight into the mechanisms which drive disease progression prior to the onset of classical PD-associated motor symptoms.

Dysregulation in the immune system has long been implicated in the pathogenesis of PD⁵, and this has led to the emergence of peripheral immune dysfunction as a promising mechanism with potentially disease-relevant biomarkers. Studies have reported increased tumor necrosis factor (TNF) receptor expression as well as enhanced production of interferon-gamma (IFNγ) and TNF from T cells in PD patients⁶. Furthermore, circulating monocytes from PD patients display upregulation of genes involved in immune activation, including HLA-DQB1, MYD88, REL, and TNF⁷. Together, these findings point towards widespread changes across both the innate and adaptive peripheral immune system in PD. Indeed, it has been shown that the neutrophil-to-lymphocyte ratio (NLR), which serves as a biomarker for systemic inflammation, is significantly correlated with lower levels of dopamine transporter in the striatum of PD patients⁸. Thus, peripheral immune dysregulation may be critical to PD progression, and targeting these mechanisms may improve the ability to deliver personalized treatment plans.

Recent meta-analyses have reported increased blood levels of inflammatory cytokines, such as TNF, IL-1β, and IL-6, in idiopathic PD (iPD) patients compared with controls^{9, 10}. It has also been found that carriers of PD-associated mutations in LRRK2 and GBA1 exhibit increased serum cytokine levels^{11, 12}, highlighting peripheral immune dysfunction as a common theme shared by both idiopathic and genetic forms of PD. However, circulating cytokine levels are subject to significant variability influenced by circadian rhythm, diet, and environmental exposures^{13, 14, 15, 16}, which has limited their effectiveness as biomarkers and contributed to heterogeneous reports^{17, 18}. Moreover, investigations of plasma cytokine levels in prodromal PD have been inconclusive. One study described increased serum TNF levels in RBD patients¹⁹, while another reported no differences relative to controls²⁰. Therefore, the extent of detectable immune dysfunction in prodromal PD remains to be determined. To facilitate the development of accurate and predictive biomarkers, new approaches are required to overcome background noise with sufficient sensitivity to reveal facets of immune dysfunction that may be difficult to parse apart at baseline.

One approach demonstrated here to help overcome these challenges is to examine differences in immune cell “traits” using stimulus-evoked responses of peripheral immune cells ex vivo. These traits are defined by stimulus-evoked activation and resolution responses, and they can be reproducibly elicited in a controlled ex vivo experiment regardless of exogenous factors. In contrast, the previous literature on baseline cytokine levels describes immune “states”, reflecting only the content of blood cytokines at a single timepoint when the sample is drawn and which can be highly variable^{13, 14, 15, 16, 21, 22}. Epidemiologic studies have linked exposure to environmental pathogens with increased long-term risk for developing PD²³, and this has raised the possibility that an aberrant immune response to stimulation may be more relevant for predicting PD risk than baseline levels of inflammatory factors in the blood. Stimulation-based assays have the potential to provide greater sensitivity, with a-synuclein peptide exposure shown to elicit increased TNF secretion in lymphocytes from PD patients but not from neurologically healthy controls (NHCs)²⁴.

Immune stimulation also has the advantage of increasing energetic demand²⁵, which can highlight immuno-metabolic deficits that have been strongly implicated in PD pathogenesis^{26, 27, 28}. For example, peripheral blood mononuclear cells (PBMCs) from PD patients show significantly altered mitochondrial respiratory capacity and mitochondrial membrane potential relative to controls^{29, 30}, as well as downregulation of a number of lysosome/autophagy related genes including ULK3, ATG2A, and HDAC6³¹. Furthermore, PD monocytes have reduced mitochondrial content relative to controls³², and monocyte activity of the lysosomal enzyme glucocerebrosidase is inversely correlated with severity of motor symptoms after diagnosis³³. Mitochondrial and lysosomal deficits have yet to be reported in PBMCs at the prodromal stage of PD, but it remains possible that metabolic organelle dysfunction is present before motor symptoms manifest yet is too subtle to observe with baseline measurements. It is therefore vital to explore how the stage of PD progression may alter immuno-metabolic response to activation, as this will not only bolster the understanding of PD etiology but potentially provide novel means of identifying at-risk individuals to recruit into suitable trials and for monitoring disease progression.

To close these important gaps in knowledge, herein it was sought to investigate whether the peripheral immune response to stimulation is dysregulated in RBD patients relative to multiple stages of PD progression. To test this hypothesis, the stimulation-dependent responses of isolated T cells and monocytes from RBD patients, iPD patients at early (within 2 years of diagnosis) and moderate (2-10 years after diagnosis) stage disease, and NHCs were studied. The inclusion of early and moderate iPD groups enabled us to capture the dynamic changes in immuno-metabolic responses across the disease spectrum. In addition, cell-type specific cytokine secretion was evaluated to enhance the ability to detect immune dysfunction traits and determine for the first time if different PBMC subsets display unique patterns of dysregulation in prodromal versus motor PD. The results show that RBD patients display a distinct signature of immune activation relative to NHCs and clinically diagnosed PD patients, and immuno-metabolic function of PBMC subsets enables stratification of PD progression across multiple stages of disease.

Therefore, based on the results here, a new method is provided to diagnose if a subject is at risk of developing Parkinson's disease (PD). In one example, the method may comprise one or more steps for obtaining and processing immune cells from a subject, such as: i. collecting blood from a subject; ii. isolating peripheral blood mononuclear cells (PBMCs) for cryopreservation and cryorecovery; iii. isolating monocytes from the PBMC, and optionally further isolating subtypes of monocytes, wherein the subtypes comprise classical monocytes (CD14+/CD16−), intermediate monocytes (CD14+/CD16+), and non-classical (CD14dim/CD16+) monocytes; iv. plating a certain number of monocytes or subtypes, and treating them with vehicle or human IFNγ, optionally for about 72 hours at about 37° C.; v. isolating CD³+ T cells from the PBMC, and optionally further isolating subtypes of T cells, wherein the subtypes comprise CD4+ T cells, CD8+ T cells, and CD8dim T cells; and/or vi. plating a certain number of CD3⁺ T cells or subtypes, and treating them with vehicle or CD3/CD28, optionally CD3/CD28 coated on magnetic beads, and optionally for about 72 hours at about 37° C. The method may further comprise steps of measuring ex vivo stimulation-evoked changes in cytokine secretion; monocyte- or T cell subtype population frequencies; the quantity of the mitochondria with negative membrane potential (healthy mitochondria) and total mitochondria, to determine the ratio of the healthy mitochondria; lysosomal function, which is assayed by measuring lysosomal cathepsin activity, and total lysosomal quantity, to determine the ratio of the functional lysosome; and changes in LRRK2 activity and LRRK2 protein level. Further a kit comprising necessary agents for such test and a diagnostic method are also suggested.

### BRIEF DESCRIPTION OF THE DRAWINGS

FIG. 1. Summary of immune dysregulation in Parkinson's disease (PD)

FIG. 2. Workflow and experimental design. Whole blood was collected from participants consisting of neurologically healthy controls (NHCs), patients with REM sleep behavior disorder (RBD), early-stage PD patients (diagnosed<2 years prior), and moderate-stage PD patients (diagnosed 2-10 years prior). Peripheral blood mononuclear cells were isolated from whole blood, cryopreserved, and thawed, and subjected to magnetic bead isolations to obtain purified CD3+ T cells or purified pan-monocytes. Cells were allowed to rest for 2 hours, followed by 72-hour incubation in presence or absence of a stimulation source (200U IFNγ for monocytes, 3.125 μL of CD3/CD28 Dynabeads for T cells). Then cells were assessed via flow cytometry and media was taken for cytokine quantification. Panel was created with BioRender.com.

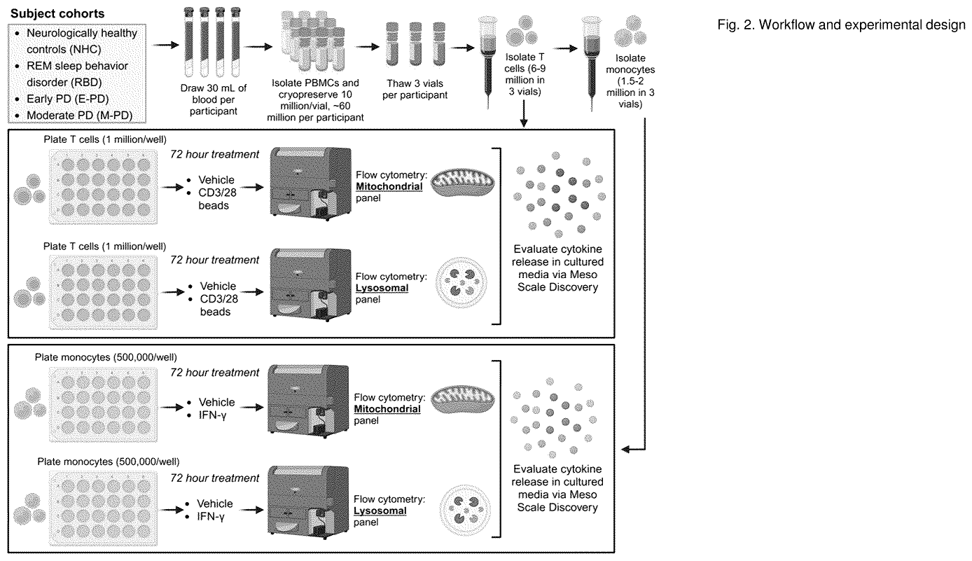

FIG. 3. Monocyte absolute cytokine secretion. Bar graphs overlaid with scatter plots showing absolute concentrations of secreted inflammatory cytokines from isolated monocytes from NHCs, RBD patients, EPD patients, and MPD patients. Absolute concentrations of secreted TNF (FIG. 3A), IL-1β (FIG. 3B), IL-8 (FIG. 3C), and IL-10 (FIG. 3D). Bars represent mean +/−SEM. NHC neurologically healthy controls, n=21; RBD patients with REM sleep behavior disorder, n=15; EPD patients with early stage PD, n=27; MPD patients with moderate stage PD, n=30. Each symbol represents the measurement from a single individual. The results in A-D were analyzed using two-way ANOVA with Tukey's corrections for multiple comparisons. Groups sharing the same letters are not significantly different (p>0.05) whilst groups displaying different letters are significantly different (p<0.05).

FIG. 4. RBD patient monocytes exhibit increased stimulation-dependent cytokine secretion. Bar graphs overlaid with scatter plots showing stimulation-dependent secretion of inflammatory cytokines after IFNγ treatment from isolated monocytes from NHCs, RBD patients, EPD patients, and MPD patients. The stimulation-dependent secretion was quantified by normalizing the absolute concentration released in the stimulated condition to the absolute concentration released in vehicle for each participant. Stimulation-dependent secretion of TNF (FIG. 4A), IL-1β (FIG. 4B), IL-8 (FIG. 4C), and IL-10 (FIG. 4D). Bars represent mean +/−SEM. NHC neurologically healthy controls, n=21; RBD patients with REM sleep behavior disorder, n=15; EPD patients with early-stage PD, n=27; MPD patients with moderate-stage PD, n=30. Each symbol represents the measurement from a single individual. The results in FIG. 4A-D were analyzed using one-way ANOVA with Tukey's corrections for multiple comparisons. Groups sharing the same letters are not significantly different (p>0.05) whilst groups displaying different letters are significantly different (p<0.05).

FIG. 5. T cell absolute cytokine secretion. Bar graphs overlaid with scatter plots showing absolute concentrations of secreted inflammatory cytokines from isolated CD3+ T cells from NHCs, RBD patients, EPD patients, and MPD patients. Absolute concentrations of secreted TNF (FIG. 5A), IL-1β (FIG. 5B), IL-8 (FIG. 5C), and IL-10 (FIG. 5D). Bars represent mean +/−SEM. NHC neurologically healthy controls, n=21; RBD patients with REM sleep behavior disorder, n=15; EPD patients with early stage PD, n=27; MPD patients with moderate stage PD, n=30. Each symbol represents the measurement from a single individual. The results in FIG. 5A-D were analyzed using two-way ANOVA with Tukey's corrections for multiple comparisons. Groups sharing the same letters are not significantly different (p>0.05) whilst groups displaying different letters are significantly different (p<0.05).

FIG. 6. T cell stimulation-dependent cytokine secretion shows diminished response in moderate PD relative to RBD. Bar graphs overlaid with scatter plots showing stimulation-dependent secretion of inflammatory cytokines from isolated T lymphocytes after CD3/CD28 treatment from NHCs, RBD patients, EPD patients, and MPD patients. The stimulation-dependent secretion was quantified by normalizing the absolute concentration released in the stimulated condition to the absolute concentration released in vehicle for each participant. Stimulation-dependent secretion of TNF (FIG. 6A), IL-1β (FIG. 6B), IL-8 (FIG. 6C), IL-2 (FIG. 6D), and IL-10 (FIG. 6E). Bars represent mean +/−SEM. NHC neurologically healthy controls, n=21; RBD patients with REM sleep behavior disorder, n=15; EPD patients with early-stage PD, n=27; MPD patients with moderate-stage PD, n=30. Each symbol represents the measurement from a single individual. The results in FIG. 6A-E were analyzed using one-way ANOVA with Tukey's corrections for multiple comparisons. Groups sharing the same letters are not significantly different (p>0.05) whilst groups displaying different letters are significantly different (p<0.05).

FIG. 7. Flow cytometry gating strategy for T cells using fluorescence-minus-one controls. Total PBMCs were stained with antibody-fluorophore conjugates and gates were defined for positive and negative populations based on <1% negatively stained cells in FMOC.

FIG. 8. Flow cytometry gating strategy for monocytes using fluorescence-minus-one controls. Isolated monocytes were stained with antibody-fluorophore conjugates and gates were defined for positive and negative populations based on <1% negatively stained cells in FMOC.

FIG. 9. PBMC subtype frequencies across multiple stages of PD. Bar graphs overlaid with scatter plots showing the frequency of subtypes of monocytes and T cells in PBMCs from NHCs, RBD patients, EPD patients, and MPD patients. FIG. 9A. Frequency of classical monocytes (CD14⁺CD16⁻) among total monocytes. FIG. 9B. Frequency of intermediate monocytes (CD14⁺CD16⁺) among total monocytes. FIG. 9C. Frequency of nonclassical monocytes (CD14^{dim}CD16⁺) among total monocytes. FIG. 9D. Frequency of CD4⁺CD8⁻ among total CD3⁺ T lymphocytes. FIG. 9E. Frequency of CD4⁻CD8⁺ among total CD3⁺T lymphocytes. FIG. 9F. Frequency of CD4⁻CD8^{dim} among total CD3⁺ T lymphocytes. Bars represent mean +/−SEM. NHC neurologically healthy controls, n=21; RBD patients with REM sleep behavior disorder, n=15; EPD patients with early-stage PD, n=27; MPD patients with moderate-stage PD, n=30. Each symbol represents the measurement from a single individual. The results in FIG. 9A-F were analyzed using two-way ANOVA with Tukey's corrections for multiple comparisons. Groups sharing the same letters are not significantly different (p>0.05) whilst groups displaying different letters are significantly different (p<0.05).

FIG. 10. PBMC subtype counts. Bar graphs overlaid with scatter plots showing the raw counts of monocytes and T cells in PBMCs from NHCs, RBD patients, EPD patients, and MPD patients. FIG. 10A. Raw counts of total monocytes (CD14+ and/or CD16+) among live cells. FIG. 10B. Raw counts of classical monocytes (CD14+CD16−) among total monocytes. FIG. 10C. Raw counts of intermediate monocytes (CD14+CD16+) among total monocytes. FIG. 10D. Raw counts of nonclassical monocytes (CD14dimCD16+) among total monocytes. FIG. 10E. Raw counts of total CD3+ T cells among total live cells. FIG. 10F. Raw counts of CD4+CD8− among total CD3+ T lymphocytes. FIG. 10G. Raw counts of CD4-CD8+ among total CD3+ T lymphocytes. FIG. 10H. Raw counts of CD4-CD8dim among total CD3+ T lymphocytes. Bars represent mean +/−SEM. NHC neurologically healthy controls, n=21; RBD patients with REM sleep behavior disorder, n=15; EPD patients with early stage PD, n=27; MPD patients with moderate stage PD, n=30. Each symbol represents the measurement from a single individual. The results in A-H were analyzed using two-way ANOVA with Tukey's corrections for multiple comparisons. Groups sharing the same letters are not significantly different (p>0.05) whilst groups displaying different letters are significantly different (p<0.05). Samples were not run to completion through flow cytometer, therefore these counts do not represent the total number of cells collected.

FIG. 11. Cytotoxic T cells from moderate PD patients show impaired relative mitochondrial health following immune stimulation. Bar graphs overlaid with scatter plots showing the mitochondrial content and mitochondrial health after CD3/CD28 stimulation of T cell subsets from NHCs, RBD patients, EPD patients, and MPD patients. FIG. 11A. Total mitochondrial content of CD8⁺ T lymphocytes. FIG. 11B. Healthy mitochondrial content with negative membrane potential in CD8⁺ T lymphocytes. FIG. 11C. Ratio of healthy mitochondrial content divided by the total in CD8⁺ T lymphocytes. FIG. 11D. Total mitochondrial content of CD8^{dim} T lymphocytes. FIG. 11E. Healthy mitochondrial content with negative membrane potential in CD8^{dim} T lymphocytes. F Ratio of healthy mitochondrial content divided by the total in CD8^{dim} T lymphocytes. FIG. 11G. Total mitochondrial content of CD4⁺ T lymphocytes. FIG. 11H. Healthy mitochondrial content with negative membrane potential in CD4⁺ T lymphocytes. FIG. 11I. Ratio of healthy mitochondrial content divided by the total in CD4⁺ T lymphocytes. Bars represent mean +/−SEM. NHC neurologically healthy controls, n=21; RBD patients with REM sleep behavior disorder, n=15; EPD patients with early-stage PD, n=27; MPD patients with moderate-stage PD, n=30. Each symbol represents the measurement from a single individual. The results in FIG. 11A-I were analyzed using two-way ANOVA with Tukey's corrections for multiple comparisons. Groups sharing the same letters are not significantly different (p>0.05) whilst groups displaying different letters are significantly different (p<0.05). MTG MitoTracker Green FM, MTR MitoTracker Red CMXRos.

FIG. 12. Monocyte mitochondrial health across multiple stages of PD. Bar graphs overlaid with scatter plots showing the mitochondrial content and mitochondrial health after immune stimulation of monocyte subsets from NHCs, RBD patients, EPD patients, and MPD patients. FIG. 12A. Total mitochondrial content of classical monocytes (CD14+CD16−). FIG. 12B. Healthy mitochondrial content with negative membrane potential in classical monocytes (CD14+CD16−). FIG. 12C. Ratio of healthy mitochondrial content divided by the total in classical monocytes (CD14+CD16−). FIG. 12D. Total mitochondrial content of intermediate monocytes (CD14+CD16+). FIG. 12E. Healthy mitochondrial content with negative membrane potential in intermediate monocytes (CD14+CD16+). FIG. 12F. Ratio of healthy mitochondrial content divided by the total in intermediate monocytes (CD14+CD16+). FIG. 12G. Total mitochondrial content of nonclassical monocytes (CD14dimCD16+). FIG. 12H. Healthy mitochondrial content with negative membrane potential in nonclassical monocytes (CD14dimCD16+). FIG. 12I. Ratio of healthy mitochondrial content divided by the total in nonclassical monocytes (CD14dimCD16+). Bars represent mean +/−SEM. NHC neurologically healthy controls, n=21; RBD patients with REM sleep behavior disorder, n=15; EPD patients with early stage PD, n=27; MPD patients with moderate stage PD, n=30. Each symbol represents the measurement from a single individual. The results in FIG. 12A-I were analyzed using two-way ANOVA with Tukey's corrections for multiple comparisons. Groups sharing the same letters are not significantly different (p>0.05) whilst groups displaying different letters are significantly different (p<0.05). MTG MitoTracker Green FM, MTR MitoTracker Red CMXRos.

FIG. 13. Relative mitochondrial health in CD8+ T cells is positively correlated with stimulation-dependent cytokine secretion from T cells in disease. Correlational analysis showing the relationship between relative mitochondrial health of CD8⁺ T cells and stimulation-dependent cytokine secretion from total T cells in RBD patients, EPD patients, and MPD patients. Correlation between relative mitochondrial health after CD3/CD28 Dynabead stimulation and stimulation-dependent secretion of TNF (FIG. 13A), IL-8 (FIG. 13B), IL-2 (FIG. 13C), IL-10 (FIG. 13D), and IL-1β(FIG. 13E). Coefficient and p-value based on Pearson correlation. Each symbol represents the measurement from a single individual. RBD patients with REM sleep behavior disorder, n=15; EPD patients with early-stage PD, n=27; MPD patients with moderate-stage PD, n=30. * signifies that the slope of the line is significantly different from zero (p<0.05).

FIG. 14. Lysosomal content of monocyte and T cell subsets across multiple stages of PD. Bar graphs overlaid with scatter plots showing the lysosomal content after immune stimulation of monocyte and T cell subsets from NHCs, RBD patients, EPD patients, and MPD patients. Lysosomal content was quantified using MFI of Lysotracker Red DND. FIG. 14A. Total lysosomal content of classical monocytes (CD14+CD16−). FIG. 14B. Total lysosomal content of intermediate monocytes (CD14+CD16+). FIG. 14C. Total lysosomal content of nonclassical monocytes (CD14dimCD16+). FIG. 14D. Total lysosomal content of CD4+/CD8− T cells. FIG. 14E. Total lysosomal content of CD4−/CD8+ T cells. FIG. 14F. Total lysosomal content of CD4−/CD8dim T cells. Bars represent mean +/−SEM. NHC neurologically healthy controls, n=21;RBD patients with REM sleep behavior disorder, n=15; EPD patients with early stage PD, n=27; MPD patients with moderate stage PD, n=30. Each symbol represents the measurement from a single individual. The results in FIG. 14A-F were analyzed using two-way ANOVA with Tukey's corrections for multiple comparisons. Groups sharing the same letters are not significantly different (p>0.05) whilst groups displaying different letters are significantly different (p<0.05). MFI median fluorescence intensity.

FIG. 15. Cathepsin activity in T cell subsets. Bar graphs overlaid with scatter plots showing the cathepsin activity after CD3/CD28 Dynabead stimulation of T cell subsets from NHCs, RBD patients, EPD patients, and MPD patients. Cathepsin activity was quantified using MFI of BMV109. FIG. 15A. Cathepsin activity of CD4+/CD8− T cells. FIG. 15B. Cathepsin activity of CD4-/CD8+ T cells. FIG. 15C. Cathepsin activity of CD4−/CD8dim T cells. Bars represent mean +/−SEM. NHC neurologically healthy controls, n=21; RBD patients with REM sleep behavior disorder, n=15; EPD patients with early stage PD, n=27; MPD patients with moderate stage PD, n=30. Each symbol represents the measurement from a single individual. The results in FIG. 15A-C were analyzed using two-way ANOVA with Tukey's corrections for multiple comparisons. Groups sharing the same letters are not significantly different (p>0.05) whilst groups displaying different letters are significantly different (p<0.05). MFI median fluorescence intensity.

FIG. 16. Monocyte subpopulations display distinct profiles of pan-cathepsin and LRRK2 kinase activity based on PD stage. Bar graphs overlaid with scatter plots showing the lysosomal pan-cathepsin activity and LRRK2 kinase activity in monocyte subtypes after IFNg treatment from NHCs, RBD patients, EPD patients, and MPD patients. FIG. 16A. Pan-cathepsin activity of classical monocytes (CD14⁺CD16⁻). FIG. 16B. Pan-cathepsin activity of intermediate monocytes (CD14⁺CD16⁺). FIG. 16C. Pan-cathepsin activity of nonclassical monocytes (CD14^{dim}CD16⁺). FIG. 16D. pRab10 expression normalized to LRRK2 expression in classical monocytes (CD14⁺CD16⁻). FIG. 16E. pRab10 expression normalized to LRRK2 expression in intermediate monocytes (CD14⁺CD16⁺). FIG. 16F. pRab10 expression normalized to LRRK2 expression in nonclassical monocytes (CD14^{dim}CD16⁺). Bars represent mean +/−SEM. NHC neurologically healthy controls, n=21; RBD patients with REM sleep behavior disorder, n=15;EPD patients with early-stage PD, n=27; MPD patients with moderate-stage PD, n=30. Each symbol represents the measurement from a single individual. The results in FIG. 16A-F were analyzed using two-way ANOVA with Tukey's corrections for multiple comparisons. Groups sharing the same letters are not significantly different (p>0.05) whilst groups displaying different letters are significantly different (p<0.05). pRab10 phosphorylated Rab10, LRRK2 leucine-rich repeat kinase 2.

FIG. 17. LRRK2 expression and pRab10 expression in monocyte subsets. Bar graphs overlaid with scatter plots showing the expression of LRRK2 and pRab10 after IFNγ stimulation of monocyte subsets from NHCs, RBD patients, EPD patients, and MPD patients. FIG. 17A. LRRK2 expression in classical monocytes (CD14+CD16−). FIG. 17B. LRRK2 expression in intermediate monocytes (CD14+CD16+). FIG. 17C. LRRK2 expression in nonclassical monocytes (CD14dimCD16+). FIG. 17D. pRab10 expression in classical monocytes (CD14+CD16−). FIG. 17E. pRab10 expression in intermediate monocytes (CD14+CD16+). FIG. 17F. pRab10 expression in nonclassical monocytes (CD14dimCD16+). Bars represent mean +/−SEM. NHC neurologically healthy controls, n=21; RBD patients with REM sleep behavior disorder, n=15; EPD patients with early stage PD, n=27; MPD patients with moderate stage PD, n=30. Each symbol represents the measurement from a single individual. The results in FIG. 17A-F were analyzed using two-way ANOVA with Tukey's corrections for multiple comparisons. Groups sharing the same letters are not significantly different (p>0.05) whilst groups displaying different letters are significantly different (p<0.05). LRRK2 leucine-rich repeat kinase 2, pRab10 phosphorylated Rab10.

FIG. 18. LRRK2 expression and pRab10 expression in T cell subsets. Bar graphs overlaid with scatter plots showing the expression of LRRK2 and pRab10 after CD3/CD28 stimulation of T cell subsets from NHCs, RBD patients, EPD patients, and MPD patients. FIG. 18A. LRRK2 expression in CD4+/CD8− T cells. FIG. 17B. LRRK2 expression in CD4−/CD8+ T cells. FIG. 17C. LRRK2 expression in CD4−/CD8dim T cells. FIG. 17D. pRab10 expression in CD4+/CD8− T cells. FIG. 17E. pRab10 expression in CD4−/CD8+ T cells. FIG. 17F. pRab10 expression in CD4−/CD8dim T cells. Bars represent mean +/−SEM. NHC neurologically healthy controls, n=21; RBD patients with REM sleep behavior disorder, n=15; EPD patients with early stage PD, n=27; MPD patients with moderate stage PD, n=30. Each symbol represents the measurement from a single individual. The results in FIG. 17A-F were analyzed using two-way ANOVA with Tukey's corrections for multiple comparisons. Groups sharing the same letters are not significantly different (p>0.05) whilst groups displaying different letters are significantly different (p<0.05). LRRK2 leucine-rich repeat kinase 2, pRab10 phosphorylated Rab10.

FIG. 19. Table 1. Demographics for and clinical information for study population

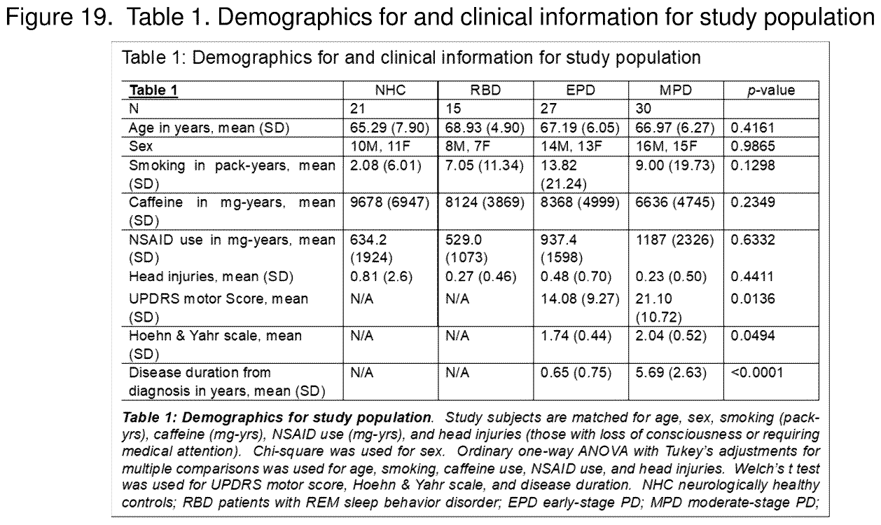

FIG. 20. Table 2. Flow cytometry T cell panel marker antibody panel; and Table 3. Flow cytometry monocyte panel marker antibody panel

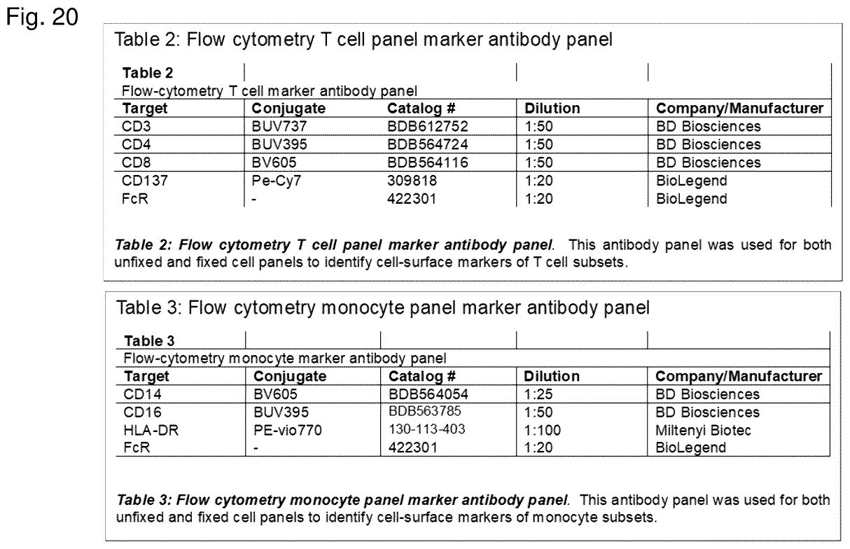

### DETAILED DESCRIPTION OF THE INVENTION

### A. Overview

Emerging literature suggests that immunophenotypic assays have the potential to more clearly define the extent of peripheral immune dysregulation in PD, thereby revealing novel biomarkers and tools for early diagnosis. However, the existence of dysregulated cytokine secretion prior to the onset of motor symptoms in PD remains a matter of contention^{19, 20}. Furthermore, studies thus far have overlooked the potential for cell-type specific differences in cytokine secretion, leaving open the possibility that specific PBMC subsets exhibit distinct patterns of immune dysfunction in PD. Here is showed that isolated monocytes and T cells from RBD patients display a distinct pattern of immune dysregulation, and that immuno-metabolic responses to stimulation stratify multiple stages of PD progression. The methodology disclosed here revealed cell-type specific differences in stimulation-evoked cytokine secretion, PBMC population composition, and facets of mitochondrial and lysosomal health which change dynamically with disease stage.

This work provides the first evidence that stimulation-evoked cytokine secretion from RBD monocytes is increased relative to NHC and PD groups. Previous studies have investigated circulating cytokine levels in RBD patients and reported heterogenous results^{19, 20}, however these studies did not examine the ex vivo response to stimulation nor did they include early or moderate iPD patients. The analysis in this disclosure suggests that baseline levels of cytokine secretion are not significantly altered in RBD monocytes, but instead that these patients display an aberrant upregulation of TNF, IL-1β, and IL-8 secretion in response to immune stimuli. This may lead to increased peripheral inflammation and represent a potential mechanism which contributes to PD conversion and progression. Support for this comes from epidemiological reports that anti-TNF therapy in inflammatory bowel disease patients and ibuprofen use in the general population are associated with lower incidence of PD in these groups^{55, 56}. Ongoing clinical trials for RBD and PD include biofluid measurements to assess inflammation 57, 58, however, it is propose here that stimulation-evoked cytokine secretion may have greater sensitivity and will more accurately report on the cell-type specificity and efficacy of immunomodulatory treatments. Thus, the inclusion of stimulation-dependent immune responses ex vivo will be necessary in future studies to directly investigate peripheral immune dysregulation as a contributing mechanism in PD pathogenesis and progression.

Concurrently, it was observed that T lymphocytes from moderate PD patients exhibit reduced secretion of TNF, IL-2, and IL-10 relative to RBD patients, a pattern suggestive of relative immune exhaustion³⁹. Given that IL-2 controls regulatory T cell maturation and proliferation⁶⁰, these results suggest that regulatory T cell maturation may be disrupted in moderate PD consistent with current literature⁶¹.

It was also shown that cytotoxic T cells in moderate PD display poorer relative mitochondrial health after stimulation compared with NHCs, and furthermore, that relative mitochondrial health after stimulation is significantly correlated with cytokine secretion. Mitochondrial impairment in neurons is widely reported in idiopathic and genetic forms of PD^{62, 63, 64}. Given that reduced mitochondrial respiration is sufficient to elicit T cell exhaustion in chronic infections⁴⁴, this may explain the immune exhaustion-like phenotype observed in moderate PD cells.

Notably, the Unified Parkinson's Disease Rating Scale (UPDRS) did not correlate with stimulation-dependent cytokine secretion, suggesting that motor severity is not sufficient to capture the underlying biology of PD as the disease progresses. Current perspectives acknowledge that additional metrics beyond motor progression are necessary to appreciate emerging sub-types of PD^{65, 66, 67}, and a combination of biochemical markers with clinical measures may be necessary to precisely identify patient endophenotypes for reduced heterogeneity in clinical trials^{68, 69}.

The analysis here revealed a hyper-inflammatory response in peripheral immune cells from RBD patients and immune exhaustion in later PD stages. Immunogenic mitochondrial damage-associated molecular patterns (DAMPs) and mitochondrial DNA are known to activate TLR4^{70, 71}, and these could be present at higher concentrations in peripheral immune cells from RBD patients. In support of this, a recent study found that fibroblasts from RBD patients who converted to PD had significantly increased mitochondrial fragmentation relative to controls, and isolated RBD patients showed similar but milder alterations⁷². Therefore, it is postulated that mitochondrial fragmentation predisposes towards a hyperinflammatory response early in disease course, but persistent accumulation of mitochondrial dysfunction eventually overwhelms the adaptive ability of these cells and leads to immune exhaustion later in PD. Future research should seek to evaluate mitochondrial morphology in RBD and PD immune cells, as pharmacologic interventions aimed at improving mitochondrial biogenesis and enhancing mitophagy represent novel and exciting avenues that should be explored to delay disease progression.

Flow cytometry analysis revealed that moderate PD patients show a reduced frequency of classical monocytes and increased frequency of intermediate monocytes compared with NHCs. The findings here are consistent with those from Thome et al. who reported identical changes in monocyte subtype frequencies in PD patients⁶¹. Intermediate monocytes have been reported to secrete higher levels of proinflammatory cytokines than classical monocytes in some contexts^{73, 74}, which seems at odds with the findings here that showed moderate PD monocytes have diminished stimulation-dependent cytokine secretion relative to RBD. One potential explanation is that the shift towards increased numbers of intermediate monocytes is a compensatory response to underlying functional deficits in these individuals.

In addition, it was observed that immune stimulation of T lymphocytes led to an increased frequency of CD8^{dim} cells in most groups; however this only reached statistical significance in moderate PD patients. Downregulation of CD8 in T lymphocytes has been reported following exposure to viral and bacterial antigens³⁹ and has been hypothesized to promote peripheral tolerance^{40, 41, 42}. Consequently, moderate PD may be associated with increased peripheral tolerance in cytotoxic T cells after immune stimulation. The data here so far cannot differentiate more specialized cell types such as helper, regulatory, or natural killer T cells, which will be important to comprehensively describe how PD stage affects peripheral immune populations. Thus, further efforts using scRNAseq or similar methodologies capable of garnering deep cell-type specific data to explore differences across these PBMC subsets should be considered.

This study also demonstrated increased pan-cathepsin activity in classical and intermediate monocytes from RBD patients at baseline relative to NHCs. Upregulation of cathepsins has been reported in multiple PD models^{75, 76}, however this work provides the first evidence that lysosomal function as measured by pan-cathepsin activity is upregulated in RBD patients. Cathepsin activity is known to modulate autophagic flux⁷⁷, therefore this may represent an increased demand for lysosomal degradation and autophagy in the prodromal stage of PD. Indeed, autophagic flux increases to compensate for mitochondrial defects⁷⁸, and mitochondrial membrane potential is a known regulator of autophagic flux⁷⁹. Lysosomal and mitochondrial dynamics are extensively linked to one another, so additional research is necessary to determine if poor mitochondrial health in RBD is a downstream consequence of disturbances in lysosomal activity. In sum, these findings point to observable changes in metabolic organelle function in RBD patients, suggesting that peripheral immuno-metabolic deficits are present in the prodromal stages of PD. Moreover, these deficits could be ascertained non-invasively to stratify and enroll patients who are more likely to benefit from interventions targeting cellular metabolism.

In addition, a mild increase in stimulation-dependent LRRK2 kinase activity was observed from RBD monocytes relative to NHCs, although this did not reach statistical significance. A larger and significant increase was observed with early and moderate PD, creating an overarching pattern consisting of gradual increases in LRRK2 kinase activity as the diseases progresses. While inhibition of LRRK2 kinase activity has been proposed as a potential approach to combat neuroinflammation in iPD⁸⁰, the data here would suggest that this may be of limited benefit in modulating peripheral inflammation in RBD patients. Instead, inhibiting LRRK2 kinase activity may be more beneficial specifically for individuals carrying the G2019S gain-of-kinase mutation, because it was previously demonstrated that LRRK2-targeting anti-sense oligonucleotide therapy reduces inflammatory cytokine secretion in LRRK2-G2019S peripheral macrophages⁸¹. However, the results here cannot conclusively distinguish whether increased LRRK2 kinase activity is compensating for or contributing to immune dysregulation in the prodromal stage of iPD, therefore additional ex vivo experiments with pharmacologic inhibitors are warranted.

This work reveals immunophenotypic differences which could serve as the basis for stratification of RBD patients with prodromal PD. Individuals with prodromal PD whose signature of immune activation is distinct from patients with motor manifestations of PD would likely benefit from early immunomodulatory interventions to arrest or slow progression. However, some limitations to this strategy must be noted. The estimated risk for patients with isolated RBD to develop PD is 43%, with the next most likely course being a 25% risk for dementia with Lewy bodies². Therefore, RBD is not completely specific for prodromal PD, and our findings may represent dysregulation in a common pathway shared by multiple neurodegenerative diseases. Second, this study design is based on cross-sectional analyses and did not include longitudinal follow up or sampling for the enrolled RBD patients; thus, it cannot be conclusively determined which of these patients will ultimately convert to PD. It will be vital for future longitudinal studies to be conducted with sufficient power to replicate these findings and correlate these immune markers with eventual PD conversion rates. In addition, these findings suggest that T cells from moderate PD patients exhibit deficits in production of cytokines such as IL-2 which is generally consistent with an immune exhaustion phenotype⁵⁹. However, further evaluations are required to definitively make the distinction between immune exhaustion and senescence. In line with this, evaluations of cell-surface markers of exhaustion such as PD-1, CTLA-4, and TIM-3 are underway in parallel studies.

In summary, this work demonstrates that peripheral blood immune cells from RBD patients display a distinct pattern of stimulation-dependent immune dysregulation relative to NHCs and clinically diagnosed PD groups. These findings hold significant potential to advance both scientific understanding and clinical practice for PD. First, the use of sensitive stimulation-dependent assays and focus on cell-type specific cytokine secretion has allowed us to detect a specific pattern of peripheral immune dysregulation in RBD patients, thereby addressing inconsistencies in previous studies. Second, this finding that T cell mitochondrial health correlates with stimulation-dependent cytokine secretion across the disease spectrum reveals a novel target for therapeutic intervention. Early intervention to rescue mitochondrial deficits may help mitigate excessive inflammation in RBD patients, while in advanced stages it may be a crucial target to combat immune exhaustion or senescence. Lastly, the expanded use of ex vivo stimulation based peripheral immune cell biomarkers, such as measures of mitochondrial health, is recommended here to distinguish unique immunophenotypes for PD patients.

It is becoming increasingly apparent that disease duration and severity of motor symptoms are not sufficient to describe the spectrum of PD progression, and the inability of the field to capture this heterogeneity across patients has been a barrier to success for clinical trials. The insights from these data should open up promising avenues for future research as the field continues to search for clinically relevant biomarkers. Moreover, applying these findings to clinical practice has the potential to significantly enhance patient-centered precision medicine in IPD.

### B. Definitions

Embodiments of materials and methods are described herein; any methods and materials similar or equivalent to those described herein can be used in the practice of or testing of the invention. Unless defined otherwise, all technical and scientific terms herein have the same meaning as commonly understood by one of ordinary skill in the art to which the invention pertains. In describing and claiming the present invention, the following terminology will be used. It is also to be understood that the terminology used herein is for the purpose of describing particular embodiments only, and it is not intended to be limiting. Also, the entire teachings of any patents, patent applications or other publications referred to herein are incorporated by reference herein as if fully set forth herein.

Other than in the operating examples, or where otherwise indicated, all numbers expressing quantities of ingredients or reaction conditions used herein should be understood as modified in all instances by the term “about.” The term “about” generally refers to a range of numbers that one of skill in the art would consider equivalent to the recited value (i.e., having the same function or result). In a specific embodiment, the term “about” includes a stated numerical value as well as a value that is +/−15% of the stated numerical value. For example, about 5.75 M includes 5.75 molar as well as 6.61 M and 4.89 M, and all 1/10 values in between. In many instances, the term “about” may include numbers that are rounded to the nearest significant figure.

The present invention can “comprise” (open ended) the components of the present invention (e.g., genes or oligonucleotide probes) as well as other ingredients or elements described herein. As used herein, “comprising” means the elements recited, or their equivalent in structure or function, plus any other element or elements which are not recited. The terms “having” and “including” are also to be construed as open ended unless the context suggests otherwise.

All ranges recited herein include the endpoints, including those that recite a range “between” two values. Terms such as “about,” “generally,” “substantially,” “approximately” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value. Unless otherwise indicated, as used herein, “a” and “an” include the plural, such that, e.g., “a gene” can mean at least one gene, as well as a plurality of genes, i.e., more than one gene. As understood by one of skill in the art, the term “gene” can be used to refer to a single gene or more than one gene.

Where used herein, the term “and/or” when used in a list of two or more items means that any one of the listed characteristics can be present, or any combination of two or more of the listed characteristics can be present. For example, if a composition of the instant invention is described as containing characteristics A, B, and/or C, the composition can contain A feature alone; B alone; C alone; A and B in combination; A and C in combination; B and C in combination; or A, B, and C in combination.

As used herein, the term “subject” or “patient” refers to a human or nonhuman mammal.

As used herein, the term “subject at risk” is a subject, who has possibility of developing Parkinson's disease, optionally who has nonmotor features of prodromal or early PD, such as anosmia (i.e., complete loss of smell), sensory disturbances (e.g., pain), mood disorder (e.g., depression), sleep disturbances (e.g., fragmented sleep, RBD), autonomic disturbances (e.g., orthostatic hypotension, gastrointestinal disturbances, genitourinary disturbances, sexual dysfunction), cognitive impairment/dementia, and/or family history.

As used herein, the term “treatment” or “treat” in the context of medical meaning refers to intervention of disease, disorder, condition or one or more symptoms thereof to obtain a desired physiological and/or clinical effect. “Treatment” or “treat” includes, but is not limited to, performing a surgical intervention and/or administering one or more drugs or agents for purposes such as: inhibiting the disease, disorder, condition, or one or more symptoms thereof; slowing or delaying the progress of the disease, disorder, condition, or one or more symptoms thereof; stabilizing (i.e., not worsening) a state of the disease, disorder, condition, or one or more symptoms thereof, and relieving, palliating, alleviating, or ameliorating the severity of the disease, disorder, condition, or one or more symptoms thereof, or preventing remission, whether partial or total and whether detectable or undetectable. “Treatment,” or “treat” may not necessarily indicate complete eradication or cure of breast cancer or associated symptoms thereof.

As used herein, the term “biomarker” comprises the meaning of an indicator, which is a measurement or value which can be interpreted as sign of a disease or condition, in addition to the meaning of a biological molecule found in blood, tissues, or other bodily fluids. A biomarker or an indicator is a measurable characteristic that implies a biological process, disease, or condition.

### C. Parkinson's Disease (pd)

Parkinson's disease (PD) is a common progressive neurodegenerative disease, affecting around 1-2% of the population over the age of 65. In PD, there is a long latency between the first damage to dopaminergic neurons of the Substantia nigra and the onset of clinical symptoms. The symptoms and signs of PD usually do not develop until 70-80% of dopaminergic neurons have already been lost. Thus, identifying subjects at-risk for PD in the period between the presumed onset of dopaminergic cell loss and the appearance of clinical Parkinsonism is of major importance for early diagnosis, the development of effective neuroprotective treatment strategies, monitoring disease progression, and providing a readout for responses to therapeutic intervention.

PD is an age-related neurodegenerative disease. Generally, the average onset age of PD is about 60 years, and the incidence of PD increases with age, but there are some cases in young individuals in their twenties or younger, particularly when PD is associated with a gene mutation.

Classical or “cardinal” features of PD are resting tremor, rigidity (stiffness), bradykinesia (slowness in starting or performing movements), and gait with postural instability, and there are additional clinical features including freezing of gait, speech difficulty, swallowing impairment, as well as nonmotor features including autonomic disturbances, sensory alterations, mood disorders, sleep dysfunction, cognitive impairment, and dementia.

Pathological features of PD are degeneration of dopaminergic neurons in the substantia nigra pars compacta (SNc), decrease of striatal dopamine, and intraneural abnormal accumulations of protein called alpha-synuclein (known as Lewy bodies) in the areas of the brain involved in thinking, memory, and movement. Although the main interest of research has been focused on dopaminergic system, Lewy bodies can be involved in neuronal degeneration in cholinergic neurons of the nucleus basalis of Meynert (NBM), norepinephrine neurons of the locus coeruleus (LC), serotonin neurons in the raphe nuclei of the brainstem, and neurons of the olfactory system, cerebral hemispheres, spinal cord, and peripheral autonomic nervous system; and such “nondopaminergic” pathology of PD may be responsible for the nonmotor clinical features. Lewy body accumulation is thought to begin in the peripheral autonomic nervous system, olfactory system, and dorsal motor nucleus of the vagus nerve in the lower brainstem, and then to spread to affect the SNc and cerebral hemispheres. In addition, clinical symptoms that are not directly related to dopaminergic neurons, such as constipation, anosmia, rapid eye movement (REM) sleep behavior disorder, and cardiac denervation can precede the onset of the motor features of PD by many years, suggesting that these may an early pre-motor stage of PD. These findings imply that the degeneration of SNc dopamine neurons and the motor symptoms of PD may develop at the mid-stage of the disease.

For the diagnosis of PD, imaging of the brain dopamine system can be performed using positron emission tomography (PET) or single photon emission computed tomography (SPECT) to observe uptake of striatal dopaminergic biomarkers. In the case of patients with family history, early age of onset (<40 years), and a particular ethnic background, genetic testing can be performed in addition to imaging for refining the diagnosis of PD. Examples of genes for genetic testing are the variants of glucocerebrosidase (GBA) gene, which are associated with 5-15% of PD patients, and the variants of LRRK2 gene, which are responsible for ˜1% of typical sporadic PD. (Harrison's Principles of Internal Medicine, 21st Ed. by Loscalzo J, Fauci AS, et al., 2022)

As for the treatment of PD, levodopa is administered in combination with a peripheral DOPA decarboxylase inhibitor (e.g., carbidopa, benserazide) to prevent its metabolism to dopamine. When co-administering levodopa with a DOPA decarboxylase inhibitor, levodopa is primarily metabolized by the catechol-O-methyltransferase (COMT), and thus inhibitors of COMT (e.g. tolcapone, entacapone, opicapone) can increase the elimination half-life of levodopa and consequently enhance its availability. Also, inhibitors of monoamine oxidase type B (MAO-B) (e.g., selegiline and rasagiline), which inhibit dopamine metabolism in the brain, are used for PD treatment. In addition, dopamine receptor agonists acting directly on postsynaptic dopamine receptors (e.g., bromocriptine, pergolide, pramipexole, ropinirole, rotigotine) are also used.

Amantadine, an antiviral agent, has been reported to inhibit the effects of adenosine at adenosine A_{2A} receptors, which may inhibit D2 receptor function. Apomorphine is a morphine derivative D2 dopamine agonist, which interacts with postsynaptic D2 receptors in the caudate nucleus and putamen, and effective for the temporary relief of off-periods of akinesia on patients on optimized dopaminergic therapy. Acetylcholine blocking drugs are also used to improve the tremor and rigidity of PD, but have little effect on bradykinesia (e.g., benztropine mesylate, biperiden, orphenadrine, trihexyphenidyl). The anticonvulsant/antiseizure drug, zonisamide is used as an add-on treatment, and has efficacy in alleviating motor symptoms. (Basic & Clinical Pharmacology, 13th Ed. by Ph.D. Katzung BG. and Trevor AJ, 2015)

As for currently possible nonpharmacological experimental therapies for PD include cell based therapies (e.g., transplantation of fetal nigral dopamine cells or dopamine neurons derived from stem cells), trophic factors (to enhance neuronal growth and restore damaged neurons), and gene therapies directed against specific target genes (to enhance glucosidase, beta, acid (GBA) gene and its product glucocerebrosidase (GCase) to promote clearance of alpha-synuclein). (Harrison's Principles of Internal Medicine, 21st Ed. by Loscalzo J, Fauci AS, et al., 2022)

### D. Examples of Embodiments

As used herein, the term “peripheral blood mononuclear cell (PBMC)” refers to any peripheral blood cell having a round nucleus. PBMCs include lymphocytes (T cells, B cells, NK cells) and monocytes. Among blood cells, erythrocytes and platelets have no nuclei, and granulocytes such as neutrophils, basophils, and eosinophils have multi-lobed nuclei. The ratio of different types of cells in PBMCs may vary between individuals, but typically in humans, lymphocytes make up the majority (70-90%) of PBMCs, monocytes 10-20%, and dendritic cells 1-2%. In one aspect, it can be contemplated to extend the measurement described in EXAMPLES to other types of PBMCs, including regulatory T cells and NK cells.

It can also be contemplated to decide normal ranges of baseline quantities and/or increase/decrease ratio after ex vivo stimulation, which indicate reference ranges (i.e., normal ranges) as seen in a blood test or lab chemical test chart in a medical report, for each cytokine, each monocyte- and T cell subtype, and lysosomal and mitochondrial function, which is to be measured from a patient sample. This may be useful to help stratify multiple stages of PD progression in addition to early diagnosis of prodromal PD in RBD patients.

As for the treatment of PD at prodromal stage before disease progress, based on the findings that TNF level increases in monocytes and T cells from RBD patients, anti-TNF agents (e.g., adalimumab (Humira), certolizumab pegol (Cimzia), etanercept (Enbrel), golimumab (Simponi), and infliximab (Remicade)) can be contemplated for the treatment of prodromal PD.

In another aspect, since T cell mitochondrial health correlates with cytokine release and inflammation, the mitochondria can be a target for therapeutic intervention with agents to enhance mitochondrial function, which may mitigate excessive inflammation in RBD patients as well as disease progress to advanced stages. Examples of the agent to treat mitochondrial dysfunction include antioxidants (e.g., coenzyme Q10 (CoQ10), lipoic acid, and N-acetyl-cysteine (NAC)), vitamins (A, C, and E), amino acids and derivatives (e.g., arginine, citrulline, L-carnitine, and creatine), mitochondria-targeted compounds (e.g., MitoQ and Bendavia (SS31)), oxidative phosphorylation (OXPHOS) modulators (e.g., idebenone, OP2113, and imeglimin), mitochondrial pyruvate carrier (MPC) blockers, and combinations thereof.

In another aspect, the results show that leucine-rich repeat kinase 2 (LRRK2) is highly expressed in IFNγ-stimulated monocytes. The exact biological function of LRRK2 remains unclear, but LRRK2 activity as an upstream regulator of Rab GTPases has been implicated in the regulation of lysosome function, which is also involved in cytokine release. It was shown in a few previous publications that some missense mutations alter the activities of LRRK2 and increase the risk of developing PD, suggesting LRRK2 in immune cells may be a useful biomarker in PD diagnosis and therapeutic target.

### EXAMPLES

### Example 1. Materials and Methods

### 1.1. Human Subjects

This study was reviewed and approved by the University of Florida Institutional Review Board (IRB202002639). Participants provided written informed consent to participate. Blood was initially collected from healthy volunteers to establish and optimize assay parameters. Then 15 subjects with RBD, 27 subjects with early PD, 30 subjects with moderate PD, and 21 age-matched, neurologically normal control subjects were recruited through the Norman Fixel Institute for Neurological Diseases at the University of Florida for this study. Early PD patients were <2 years post-diagnosis with <1 year on PD medications. Moderate PD patients were between 2-10 years post-diagnosis. Subjects were excluded based on age (younger than 50 and over 85 years of age), known familial PD mutations and/or other known neurological, chronic or recent infections, or autoimmune comorbidities. Subjects were genotyped for the LRRK2-G2019S mutation (Life Technologies #4351378, Grand Island, NY) and excluded from this study if they were shown to be mutation carriers.

During recruitment, a family history and environmental questionnaire was used to assess history of disease and inflammation/immuno-relevant environmental exposures and comorbidities. Caffeine use, non-steroidal anti-inflammatory drug (NSAID) use, and nicotine exposure were calculated as mg-years, mg-years, and pack-years, respectively. The study populations were balanced with respect to risk factors for PD, including age, smoking, non-steroidal anti-inflammatory drug use, and caffeine intake (Table 1).

1.2. Peripheral Blood Mononuclear Cell (pbmc) Isolation and Cryopreservation

Cell isolation was accomplished using BD Vacutainer CPT Cell Preparation Tube with Sodium Citrate (BD Biosciences, 362761). Approximately 6 CPT tubes, each containing 8 mL of blood, were collected from each participant. CPT tubes were inverted 8-10 times and centrifuged at room temperature at 1500×g for 20 min at room temperature. The PBMC enriched layer was transferred to a new 50 ml conical tube and MACS buffer (PBS, 0.5% bovine serum albumin, 20 mM EDTA, pH 7.2) was added to a final volume of 50 mL, followed by centrifugation at 1800×g for 10 min at room temperature. Following removal of the supernatant, PBMCs were resuspended in 10 mL MACS buffer and counted on a hemocytometer using Trypan blue (1:20 dilution) exclusion to ascertain viability.

Next, to cryopreserve the samples, PBMCs were centrifuged for 5 min 1800×g at room temperature. Supernatant was aspirated and cell pellets were gently resuspended in cryopreservation media (54% RPMI 1640, 36% FBS, 10% DMSO) at a final concentration of 1×10⁷ cells/mL in cryovials (Simport, T311-2). Cryovials were placed in a room-temperature Mr. Frosty freezing container with isopropanol as per manufacturer's instructions and stored at −80° C. overnight. After overnight storage at −80° C., the next day cryovials were removed from freezing containers and placed into liquid nitrogen for long-term storage.

### 1.3. Cryorecovery of Isolated Pbmcs

For cryorecovery, cryovials of PBMCs were retrieved from liquid nitrogen, rapidly thawed in a water bath at 37° C., and rapidly added to 25 mL of 37° C. filter sterilized complete culture media (RPMI 1640 media, 10% low endotoxin heat-inactivated FBS, 1 mM Penicillin-Streptomycin). PBMCs were pelleted via centrifugation at 300×g for 10 min at room temperature. Pellets were gently resuspended in 10 mL of 37° C. MACS buffer (PBS, 0.5% bovine serum albumin, 20 mM EDTA, pH 7.2), then viability and cell count were obtained with a hemocytometer using Trypan blue (1:20 dilution) exclusion to ascertain viability.

### 1.4. Ex Vivo Isolation of CD3+ T Cells and Pan Monocytes From Pbmcs

Following cryorecovery, CD3+ T cells were isolated from total PBMCs using REAlease® CD3 MicroBead Kit, human (Miltenyi, 130-117-038) following the manufacturer's instructions with slight modifications. PBMCs were centrifuged at 300×g for 10 min at room temperature, supernatant was aspirated, and pellets were gently resuspended in 40 μL of separation buffer (PBS, 0.5% bovine serum albumin, 2 mM EDTA, pH 7.2) per 1×10⁷ total cells. 10 μL of REAlease CD3-Biotin (CD3, human) were added per 1×10⁷ total cells, mixed well, and samples were incubated at room temperature for 5 minutes. 100 μL of REAlease Anti-Biotin Microbeads (CD3, human) were added per 1×10⁷ total cells, mixed well, and samples were incubated at room temperature for 5 minutes. Samples were diluted to a total volume of 2 mL with separation buffer then passed through pre-wetted LS columns (Miltenyi, 130-042-401) in a QuadroMACS™ Separator (Miltenyi, 130-091-051). Columns were washed 3 times with 3 mL of separation buffer, and the flow-through was set aside at 4° C. for isolation of monocytes. LS columns were removed from the magnetic separator and flushed twice with 5 mL of REAlease Bead Release buffer to release bead-bound CD3⁺ cells. CD3+ samples were mixed well and incubated at room temperature for 5 minutes. Then, CD3⁺ samples were centrifuged at 300×g for 10 min at 4° C., supernatant was aspirated, pellets were gently resuspended in 5 mL separation buffer. 100 μL of REAlease Release Reagent was added to each sample, mixed well, and then CD3⁺ cells were counted using a hemocytometer with Trypan blue (1:20 dilution) to ascertain viability.

Monocytes were isolated from the flow-through of CD3⁻cells using Pan Monocyte Isolation Kit, human (Miltenyi, 130-096-537) following the manufacturer's instructions with slight modifications. Cells were centrifuged at 300×g for 10 min at 4° C., supernatant was aspirated, and pellets were gently resuspended in 45 μL of cold separation buffer per 1×10⁷ total cells. 15 μL of FcR blocking reagent and 18.75 μL of Biotin-antibody cocktail was added per 1×10⁷ total cells, samples were mixed well, and then cells were incubated for 5 minutes at 4° C. 45 μL of cold separation buffer and 30 μL of Anti-Biotin Microbeads were added per 1×10⁷ total cells, samples were mixed well, and then cells were incubated for 5 minutes at 4° C. Samples were diluted to a total volume of 2 mL with cold separation buffer then passed through pre-wetted LS columns in a QuadroMACS™ Separator. Columns were washed 3 times with 3 mL of separation buffer, and the flow-through containing purified monocytes was counted on a hemocytometer using Trypan blue (1:20 dilution) to ascertain viability.

### 1.5. Ex Vivo T Cell and Monocyte Cell Culture Plating and Treatments

T cells were diluted to a final concentration of 1×10⁶ per mL in 1 mL complete culture media in 24-well plates and allowed to rest for 2 hours at 37° C., 5% CO₂, 95% relative humidity. After resting, cells were treated with either vehicle or 3.125 μL Dynabeads™ Human T-Activator CD 3/CD28 (Gibco, 11161D) for 72 hours at 37° C., 5% CO₂, 95% relative humidity.

Monocytes were diluted to a final concentration of 5×10⁵ per mL in 1 mL complete culture media in 24-well plates and allowed to rest for 2 hours at 37° C., 5% CO₂, 95% relative humidity. After resting, cells were treated with either vehicle or 200U human IFNγ (Peprotech, 300-02) for 72 hours at 37° C., 5% CO₂, 95% relative humidity.

### 1.6. Live Cell Flow Cytometry Assay for Mitochondrial Health and Pan-cathepsin Activity

After the 72-hour stimulation, cells were harvested and centrifuged at 300×g for 10 min at 4° C. Supernatant was collected to quantify cytokine secretion (described below). Cell pellets were gently resuspended in 200 μL of cold PBS and transferred to a v-bottom 96-well plate (Sigma, CLS3896-48EA). Samples were centrifuged at 300×g for 5 min at 4° C. Cells were resuspended in 200 μL of complete growth media containing 1 μM MitoTracker™ Red CMXRos (Invitrogen, M7512), 1 μM MitoTracker™ Green FM (Invitrogen, M7514), and 1 μM BMV109Pan Cathepsin Probe (Vergent Biosciences, 40200-200). Cells were incubated for 1 hour at 37° C. in the dark. Samples were centrifuged at 300×g for 5 min at 4° C. Cell pellets were resuspended in PBS and washed×2 by centrifugation at 300×g for 5 min at 4° C. Cells were resuspended in 50 μL of Live/Dead Fixable Violet stain (diluted 1:2000 in PBS, Invitrogen, L34962) and incubated in the dark at room temperature for 30 min. Cells were centrifuged at 300×g for 5 min at 4° C. washed in PBS×2. Cells were resuspended in 50 μL of PBS containing diluted antibodies (see Table 2 for T cell panel, see Table 3 for monocyte panel) and incubated in the dark at 4° C. for 20 min. Cells were centrifuged at 300×g for 5 min at 4° C. washed in FACS buffer (PBS, 0.5 mM EDTA, 0.1% sodium azide)×3. Cells were analyzed via flow cytometry on a FACSymphony™ A3 cytometer (BD Biosciences). Data were analyzed using FlowJo version 10.10.0 software (BD Biosciences). When validating all flow cytometry panels and antibodies, fluorescence minus one controls (FMOCs) were used to set gates and isotype controls were used to ensure antibody-specific binding.

### 1.7. Fixed Cell Flow Cytometry and Staining for Lysosomal Health and Lrrk2 Activity

After the 72-hour stimulation, cells were harvested and centrifuged at 300×g for 5 min at 4° C. Supernatant was collected to quantify cytokine secretion (described below). Cell pellets were gently resuspended in 200 μL of cold PBS and transferred to a v-bottom 96-well plate (Sigma, CLS3896-48EA). Samples were centrifuged at 300×g for 5 min at 4° C. Cells were resuspended in 200 μL of complete growth media, and LysoTracker™. Red DND-99 (Invitrogen, L7528) was added to reach a final concentration of 500 nM for T cells or 200 nM for monocytes. Cells were incubated for 1 hour at 37° C. in the dark. Samples were centrifuged at 300×g for 5 min at 4° C. Cell pellets were resuspended in PBS and washed×2 by centrifugation at 300×g for 5 min at 4° C. Cells were resuspended in 50 μL of Live/Dead Fixable Violet stain (diluted 1:2000 in PBS, Invitrogen, L34962) and incubated in the dark at room temperature for 30 min. Cells were centrifuged at 300×g for 5 min at 4° C. washed in PBS×2. Cells were resuspended in 50 μL of PBS containing diluted antibodies (see Table 2 for T cell panel, see Table 3 for monocyte panel) and incubated in the dark at 4° C. for 20 min. Cells were centrifuged at 300×g for 5 min at 4° C. and washed×2 in PBS. Cells were re-suspended and fixed in 100 μL of 1% paraformaldehyde (PFA) at 4° C. in the dark for 30 min. Cells were washed 2× with PBS, then resuspended in 100 μL of permeabilization buffer (eBiosciences, 00-8333-56) and incubated on ice for 15 min. Anti-pT 73 Rab10 antibody (Abcam, ab241060) was added to each well at 0.55 ug per well and incubated at room temperature and protected from light for 30 min. Cells were centrifuged at 300 ×g for 5 min at 4° C. washed in PBS×2. Cells were resuspended in 100 ×L of PBS containing 1% normal goat/donkey serum, 2% BSA and 1:1000 AF488 donkey anti-rabbit secondary (Thermo Fisher, A-21206) and incubated at room temperature and protected from light for 30 min. Cells were centrifuged at 300×g for 5 min at 4° C. washed in PBS×2. Cells were resuspended in 100 ×L of PBS containing 1% normal goat/donkey serum, 2% BSA 1:100 anti-LRRK2 AF700 antibody and incubated at 4° C. covered for 20 min. Cells were centrifuged at 300×g for 5 min at 4° C., and then washed in FACS buffer×3. Cells were analyzed via flow cytometry on a FACSymphony™ A3 cytometer (BD Biosciences). Data were analyzed using FlowJo version 10.10.0 software.

### 1.8. Cytokine Quantification

V-PLEX custom Human Biomarkers kit (Meso Scale Discovery (MSD), K151ARH-2) was used to quantify cytokines in conditioned media from cultured T cells and monocytes. Media was diluted 1:4 with MSD kit diluent and incubated in duplicate at room temperature in the provided MSD plate with capture antibodies for 2 hours as per manufacturer's instructions. Plates were then washed×3 with PBS with 0.05% Tween-20 and detection antibodies conjugated with electrochemiluminescent labels were added and incubated at room temperature for another 2 hours. After 3× washes with PBS containing 0.05% Tween-20, MSD buffer was added, and the plates were loaded into the QuickPlex MSD instrument for quantification.

### 1.9. Statistics

Data and statistical analyses were performed using GraphPad Prism 10. For assessing differences between groups, data were analyzed by either one-way or two-way analysis of variance (ANOVA), or by t-test. In instances when data did not fit parametric assumptions, Kruskal-Wallis non-parametric ANOVA was used. Post-hoc tests following ANOVAs were conducted using Tukey's method for correcting for multiple comparisons. For assessing relationships between read-outs, data were analyzed by Pearson's r. In instances when data did not fit parametric assumptions, Spearman's rank was used to assess relationships between variables. Two-tailed levels of significance were used and p<0.05 was considered statistically significant. Graphs are depicted by means±standard error of the mean (SEM).

### Example 2. Monocytes From Rbd Patients Display Dysregulated Stimulation-Dependent Cytokine Secretion

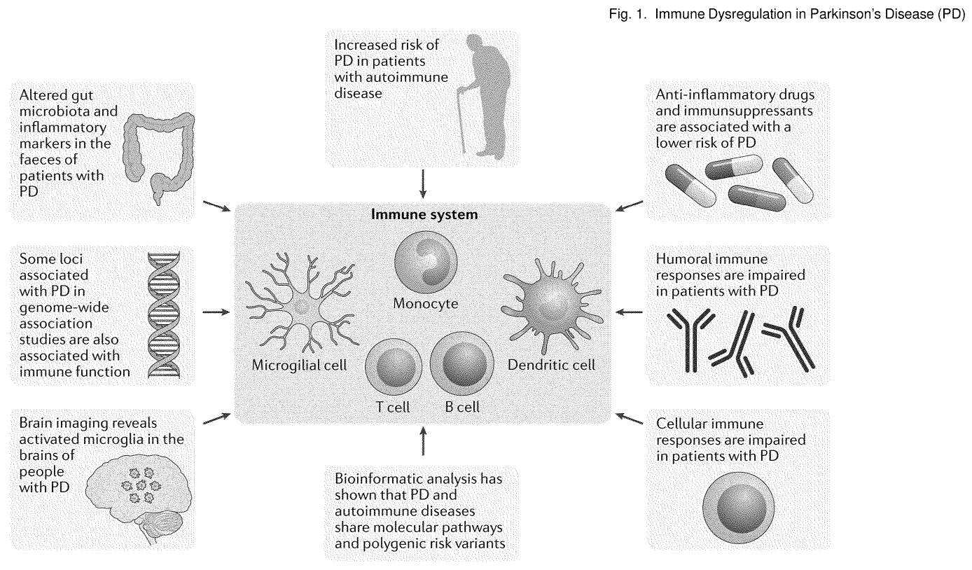

To determine if PBMC subsets from iPD patients exhibit differences in stimulation-evoked inflammatory cytokine secretion based on disease progression, PBMCs from patients across the disease spectrum were collected. Whole blood samples were collected from RBD patients to approximate prodromal PD (n=15), early-stage PD patients within 2 years of diagnosis (n=27), moderate-stage PD patients within 2-10 years of diagnosis (n=30), and age-and sex-matched neurologically healthy controls (NHC, n=21). Patients were enrolled at the Norman Fixel Institute for Neurological Diseases at the University of Florida (demographic information of the cohorts is shown in Table 1). From whole blood, PBMCs were isolated and cryopreserved using previously published methods³⁴. After PBMCs were thawed, CD3⁺ T cells and pan-monocytes were magnetically isolated, plated, and treated with an immune stimulus (200U IFNγ for monocytes and 3.125 μL of CD3/CD28 T-Activator Dynabeads for T cells) or vehicle control for 72 hours (workflow is shown in FIG. 1).

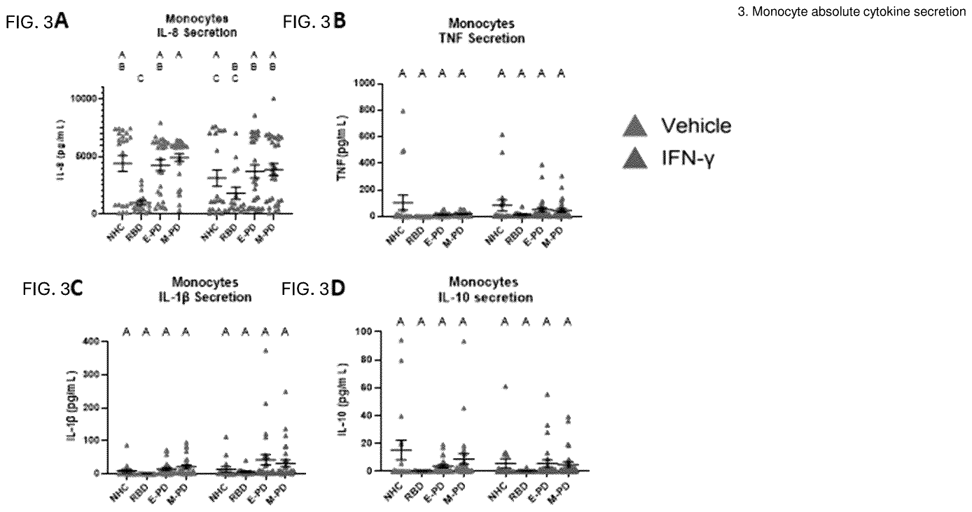

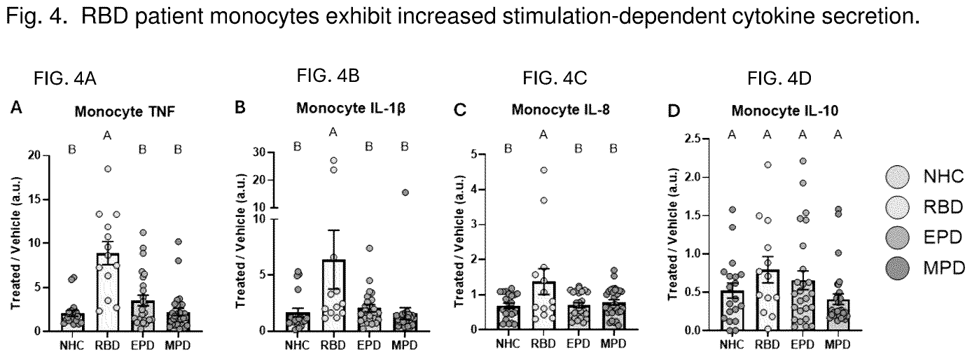

To determine the effects of PD progression on stimulation-evoked innate immune responses, the concentrations of inflammatory cytokines in the cultured media from isolated monocytes under baseline and stimulated conditions were first assessed. Using multiplexed immunoassay platform (Meso Scale Discovery), it was observed that IL-8 concentration in the media from vehicle-treated RBD monocytes was significantly reduced relative to other groups (FIG. 3A), however absolute concentrations of TNF, IL-1β, and IL-10 were not significantly affected by PD status (FIG. 3B-D). Due to significant variability in the data, the concentrations of secreted cytokines in the stimulated condition were normalized to the amount secreted in the vehicle condition, allowing each patient to serve as their own normalization factor. It was observed that monocytes from RBD patients exhibited significantly increased stimulation-dependent secretion of TNF, IL-1β, and IL-8 relative to all other groups (FIG. 4A-C). No significant differences were observed in IL-10 secretion between patient groups (FIG. 4D). Therefore, anti-inflammatory cytokine secretion was not as significantly modified by PD status as pro-inflammatory pathways. Collectively, such data support a pattern of upregulated stimulation-dependent secretion of proinflammatory cytokines by monocytes in the prodromal stage of PD, which subsequently diminishes as the disease progresses as evidenced by the levels in the early-and mid-stage PD cohorts.

### Example 3. Later Stages of PD are Characterized by Progressive Reductions in T cell Cytokine Secretion Relative to Individuals With RBD

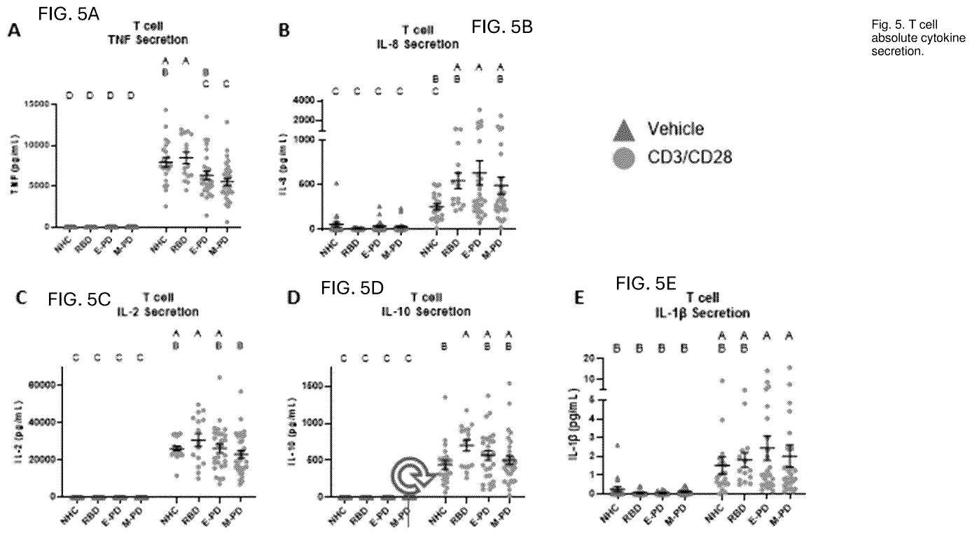

Next, it was sought to assess if isolated CD3⁺ T lymphocytes displayed similar or distinct patterns of stimulation-dependent cytokine secretion compared with those observed in monocytes. The absolute concentrations of cytokines in the cultured media of T lymphocytes were compared following treatment with vehicle or CD3/CD28 Dynabeads. Absolute levels of TNF secreted from stimulated T cells from RBD patients were significantly increased relative to those from early and moderate PD (FIG. 5A). This may indicate that the capacity for T lymphocytes to secrete TNF peaks during prodromal stages and diminishes upon the onset and progression of PD. In addition, it was observed that IL-8 secretion from stimulated T cells was highest in early PD and reached statistical significance relative to NHCs (FIG. 5B). Stimulated RBD T cells also showed increased secretion of IL-2 relative to moderate PD and increased secretion of IL-10 relative to NHC, but no differences were observed in IL-1β secretion across patient groups (FIG. 5C-E).

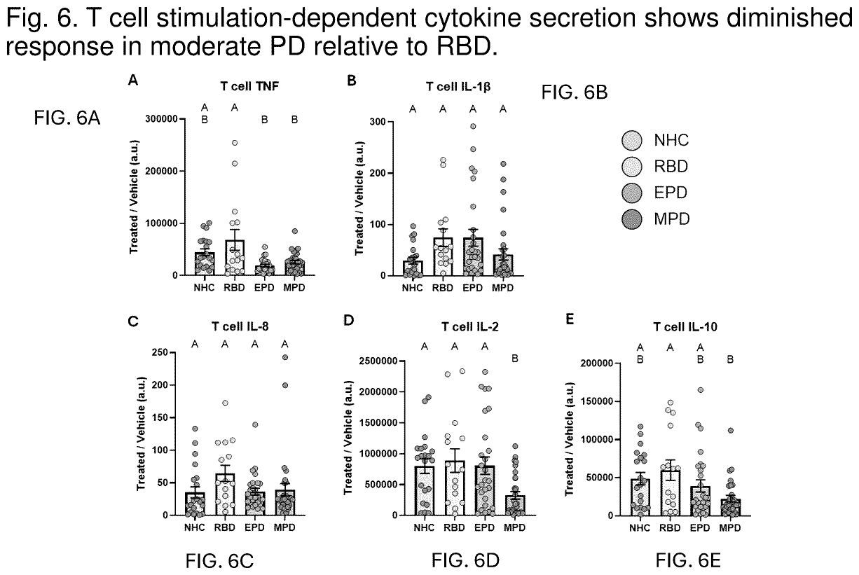

To mitigate variability, the cytokine secretion from stimulated condition was normalized to the vehicle condition. Consistent with the analysis of absolute concentrations, relative stimulation-dependent secretion of TNF was increased from RBD T cells compared with early and moderate PD groups (FIG. 6A). No differences were observed across groups in terms of relative IL-1β and IL-8 secretion (FIG. 6B, C). Interestingly stimulation-dependent secretion of IL-2 from T cells was decreased in moderate PD relative to all other groups (FIG. 6D). In addition, stimulation-dependent IL-10 secretion was reduced in moderate PD relative to RBD patients (FIG. 6E). Overall, RBD T lymphocytes displayed a pattern of hyperinflammatory response to stimulation compared with moderate PD, which progressively diminishes as the stage of PD advances, in some cases dropping below NHC levels.

### Example 4. Differences in Pbmc Subtype Frequencies Reveal Changes in Immunophenotype Based on Stage of PD

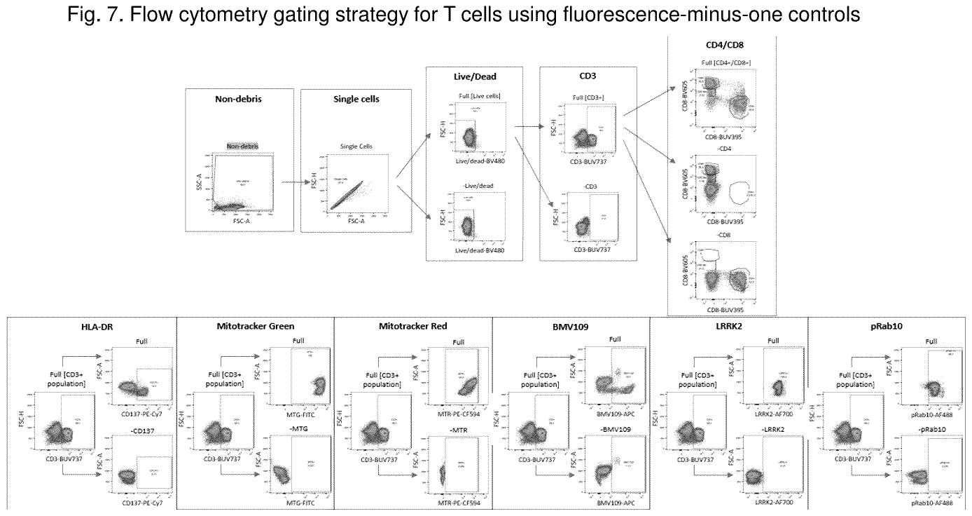

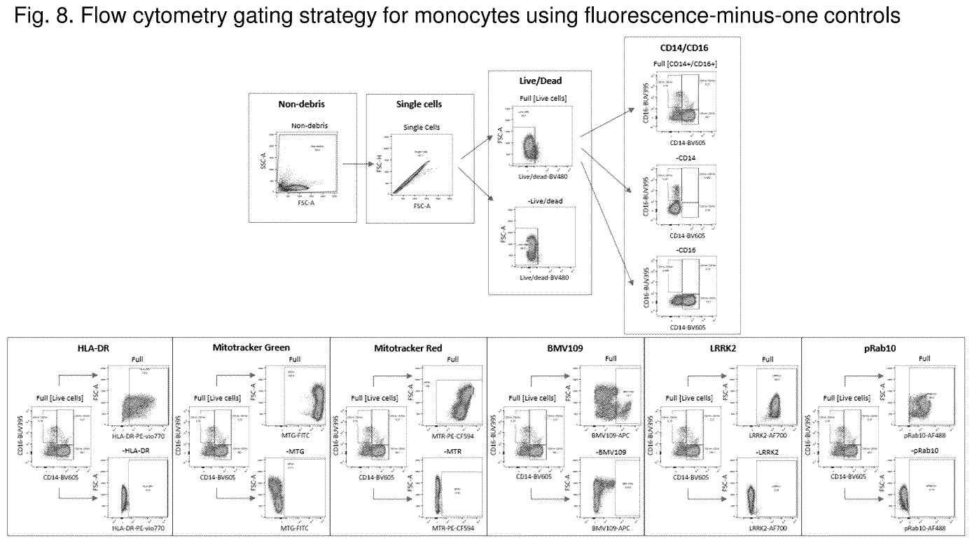

Prior studies have linked PD status to changes in the frequency of PBMC subtypes^{8, 35}, so it was sought to investigate if the observed changes in cytokine secretion were driven by changes in monocyte or T cell sub-population frequencies. To assess this, the isolated monocytes and T cells were stained after treatment for flow cytometry analysis using antibody-fluorophore conjugates for cell-surface markers (see FIGS. 7 and 8 for gating strategy with fluorescence-minus-one controls (FMOCs)).

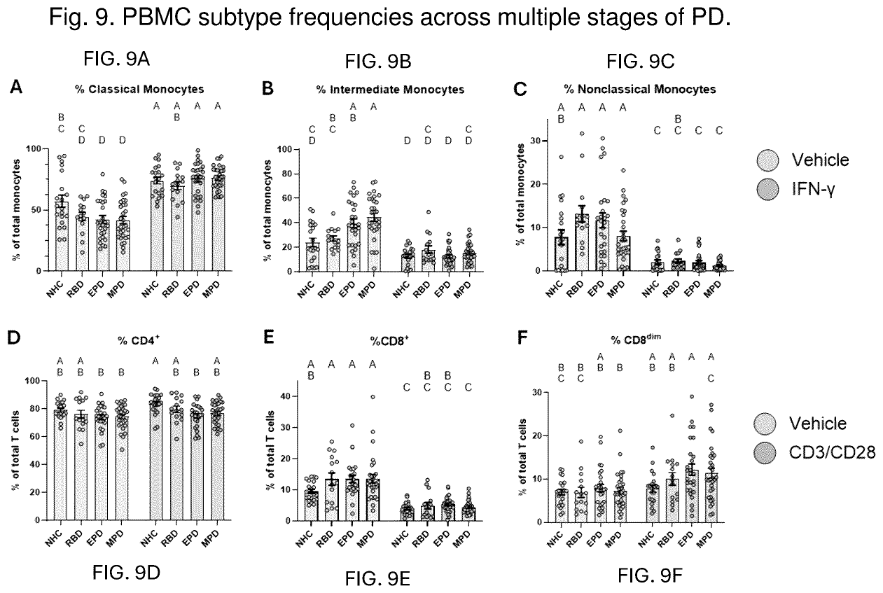

Reduced frequencies of classical monocytes (CD14+CD16−) were observed in vehicle-treated early and moderate PD groups relative to NHCs (FIG. 9A). IFNγ treatment caused an increase in the frequency of classical monocytes in all groups and ablated inter-group differences. Intriguingly, early PD patients displayed significantly elevated proportions of intermediate monocytes (CD14+CD16+) relative to NHCs in the vehicle condition (FIG. 9B). Furthermore, moderate PD patients showed higher frequencies of intermediate monocytes than both NHC and RBD groups in vehicle condition. This was surprising because earlier data from this lab suggested that early and moderate PD monocytes as a whole display weaker stimulation-dependent cytokine secretion, yet intermediate monocytes are associated with strong secretion of proinflammatory cytokines³⁶.

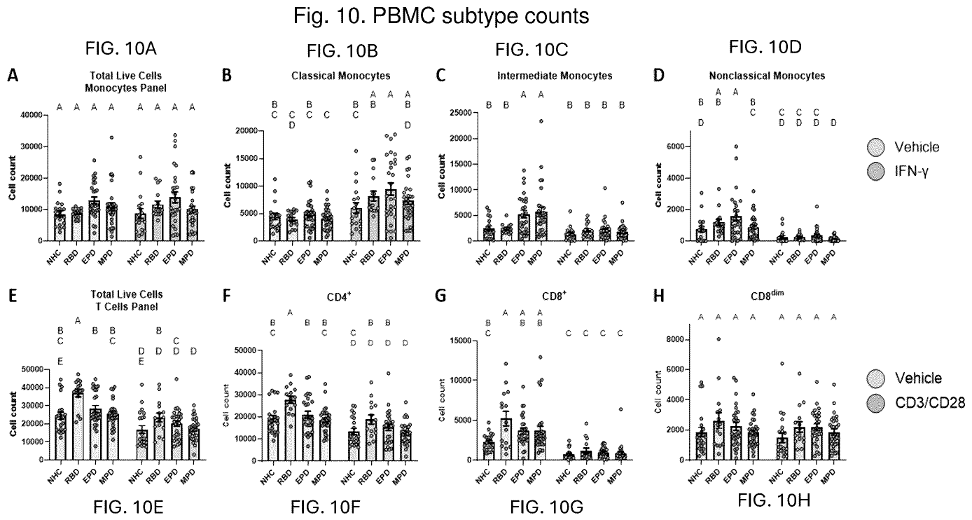

Differences across patient groups in the frequency of non-classical (CD14^{dim}CD16+) monocytes were not observed (FIG. 9C). IFNγ treatment evoked significant reductions in the frequencies of both intermediate and non-classical monocyte populations (FIG. 9B, C). These changes were not due to differences in the raw counts of total monocytes (FIG. 10). In sum, these results suggest that PD is associated with a shift towards higher frequencies of intermediate monocytes at baseline, away from classical monocytes, but PD status does not interfere with the ability of monocytes to class switch between subtypes following immune stimulation.

Next, it was evaluated if the different stages of PD progression were associated with changes in the baseline or stimulation-evoked frequencies of CD4⁺ or CD8⁺ cells among T lymphocytes. CD4⁺ T cell frequencies were similar across groups in the vehicle treated condition, however CD3/CD28 stimulation resulted in a small but statistically significant decrease in CD4+ frequency in early PD relative to NHCs (FIG. 9D). The frequency of CD8+ T cells was not significantly different across groups in vehicle or stimulated conditions, and stimulation universally led to a decrease in the fraction of CD8⁺ cells (FIG. 9E).

Also a population of CD4⁻ cells were observed, which were dimly positive for CD8, distinct from the brighter CD8+ population, and this group was termed CD8^{dim} (FIG. 7). CD8^{dim} cells have been reported by others^{37, 38} and are believed to arise after excessive pathogen burden or immune activation³⁸. Bead stimulation caused an increase in the frequency of CD8^{dim} T cells (treatment effect, p=0.0001) (FIG. 9F), however, only the moderate PD group showed a statistically significant increase in CD8^{dim} after treatment. Downregulation of CD8 in T lymphocytes has been reported following exposure to viral and bacterial antigens³⁹, and it has been hypothesized to promote peripheral tolerance^{40, 41, 42}. Therefore, the increase in CD8^{dim} T cells after stimulation supports a pattern of increased peripheral tolerance in moderate-stage PD.

### Example 5. Immune Activation Reveals Deficits in CD8⁺ T cell Mitochondrial Health in Moderate PD

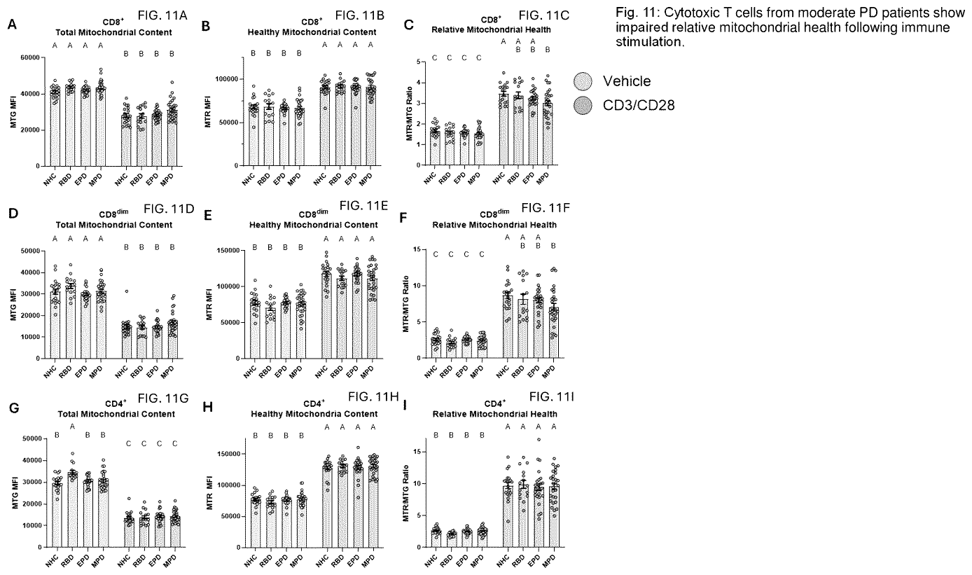

Growing evidence suggests that cellular metabolic function is intricately connected to immune responses⁴³. Given that decreased cytokine production in immune cells can be caused by mitochondrial dysfunction^{44, 45}, it was sought to assess if the stage of PD progression was associated with altered mitochondrial health in PBMC subsets. To investigate this, the median fluorescence intensity (MFI) of MitoTracker probes was quantified in stimulated PBMCs using flow cytometry. MitoTracker Green FM (MTG) was used to probe total mitochondrial content⁴⁶. Mito Tracker Red CMXRos (MTR) accumulates and stains mitochondria with sufficiently negative membrane potential⁴⁶. A negative membrane potential is required for ATP production and normal mitochondrial function⁴⁷, thus MTR MFI was used to quantify healthy mitochondrial content. It was observed that total mitochondrial content and healthy mitochondrial content were not significantly different across patient groups in CD8⁺ T cells (FIG. 11A, B).

To determine if PD progression modulates the relative proportion of healthy mitochondria within the cell, the MFI ratio of MTR/MTG was calculated, and this ratio was compared across groups. Intriguingly, stimulated CD8+ T cells from moderate PD patients showed significantly lower MTR/MTG ratio than NHCs (FIG. 11C). This effect was specific to stimulated cells, as no differences in MTR/MTG ratio were observed at baseline. In the CD8^{dim} population, the effect of PD status on total mitochondrial content or healthy mitochondrial content were not observe (FIG. 11D, E). However, CD8^{dim} lymphocytes from moderate PD patients showed significantly reduced relative mitochondrial health after stimulation (FIG. 11F), mirroring the findings from CD8+ cells. These results suggest that cytotoxic T cells from moderate PD patients are unable to maintain a high fraction of healthy mitochondria in response to immune activation and increased bioenergetic demand.

Then, CD4⁺ cells were examined, and it was observed that total mitochondrial content in RBD cells was significantly increased relative to all other groups in the vehicle condition (FIG. 11G). No differences were observed across groups in healthy mitochondrial content or relative mitochondrial health in CD4+ cells (FIG. 11H, I). These results suggest that RBD may be associated with a greater demand for mitochondria and energy production in CD4+ cells at baseline, however, RBD status does not disrupt the ability of these cells to regulate mitochondrial health in response to immune activation.

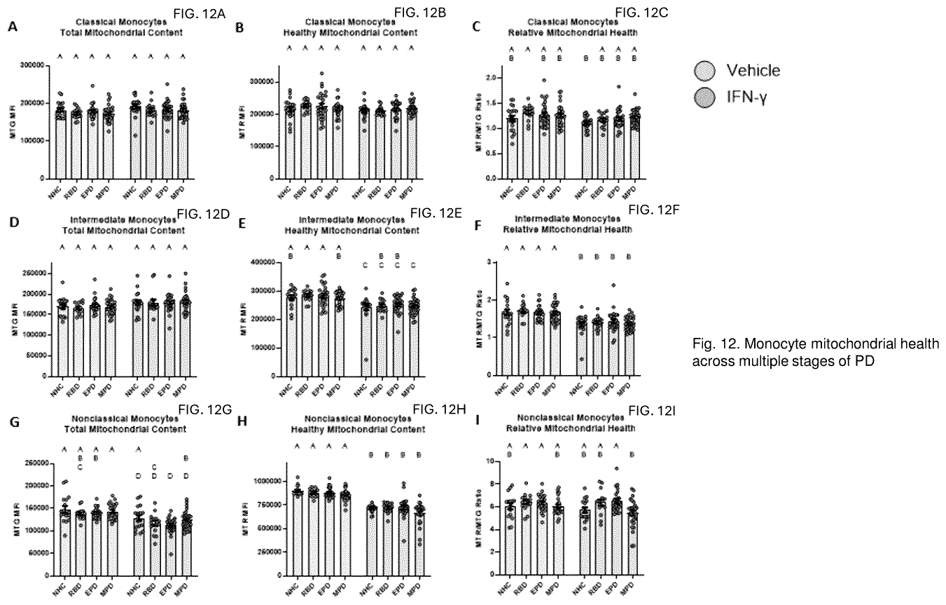

Next, mitochondrial health in monocyte subpopulations was examined, and it was observed that PD status was not associated with significant changes in mitochondrial health in classical and intermediate monocytes (FIG. 12A-F). In nonclassical monocytes, total mitochondrial content and healthy mitochondrial content also remained unaffected by PD status (FIG. 12G, H), but the relative mitochondrial health in moderate PD patients was significantly reduced compared with early PD after stimulation. These data suggest that PD status does not compromise the ability of monocytes to regulate mitochondrial health after stimulation, with the exception of nonclassical monocytes in late stages of disease.

### Example 6. Relative Mitochondrial Health in CD8⁺ T Cells is Positively Correlated With Stimulation-Dependent T cell Cytokine Secretion Across the PD Spectrum

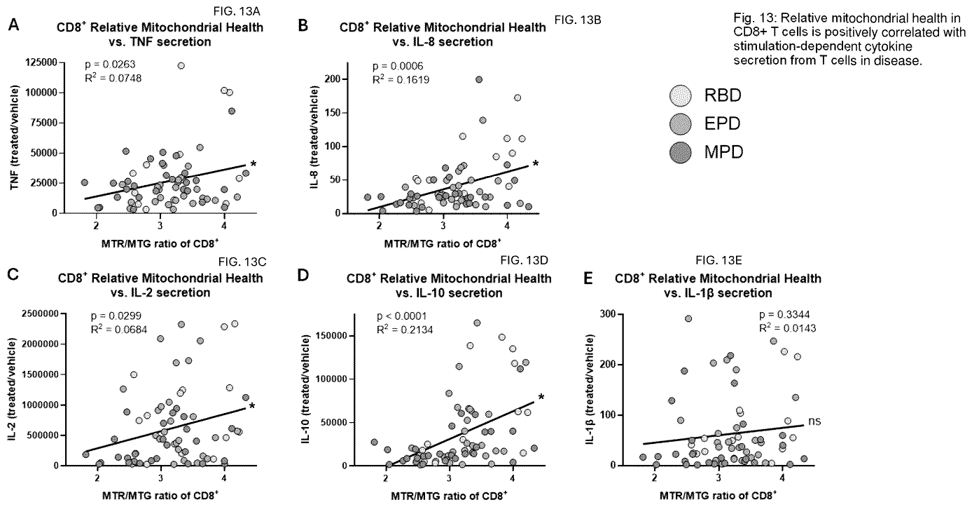

Previous reports show that mitochondrial deficits in immune cells can contribute to decreased cytokine secretion^{44, 48}. Therefore, it was sought to explore if the deficits observed in mitochondrial health subsequent to immune stimulation were correlated with deficient cytokine secretion across multiple stages of PD. It was noted that reductions in relative mitochondrial health with advanced stages of PD were specific to cytotoxic T cells, thus linear regression was used to determine the relationship between CD8⁺ MTR/MTG ratio and T cell cytokine secretion. Relative mitochondrial health was found to be significantly positively correlated with T cell stimulation-dependent secretion of TNF, IL-8, IL-2, and IL-10 (FIG. 13A-D) but not IL-1β (FIG. 13E). To ensure this relationship was not driven by motor severity, patient UPDRS scores in early and moderate PD groups were also compared with cytokine secretion, and no statistically significant correlations were observed. These results indicate that mitochondrial health may represent a useful metric for understanding peripheral immune responses across multiple stages of PD progression, including in RBD patients.

### Example 7. Monocyte Lysosomal Function and Lrrk2 Activity Differentiate Rbd From Motor Pd

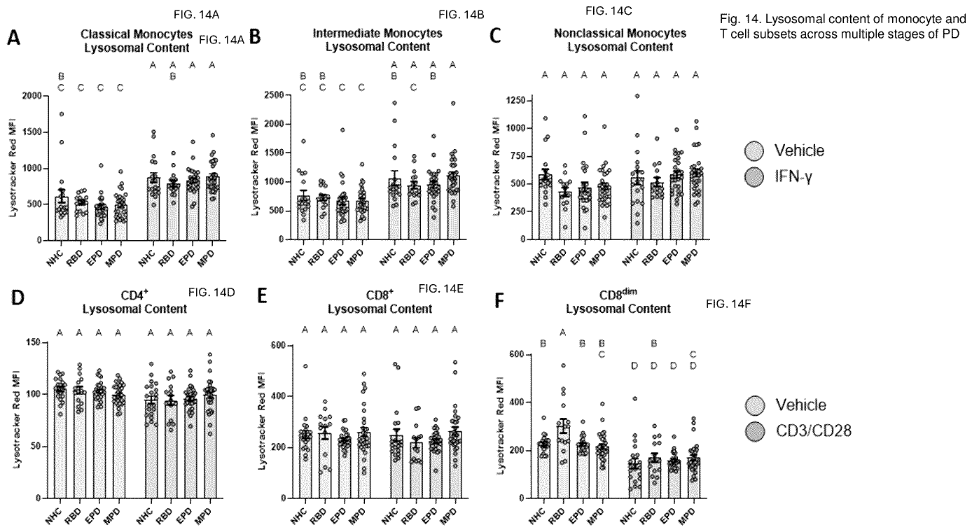

PD has been linked to deficits in lysosomal degradation in PBMCs⁴⁹, and the severity of motor symptoms in PD patients is inversely correlated with monocyte activity of the lysosomal enzyme glucocerebrosidase³³. Furthermore, pharmacological inhibition of lysosomal function polarizes immune cells towards a more proinflammatory phenotype and enhances proinflammatory cytokine production⁵⁰. This led to a question whether different stages of PD were associated with stimulation-dependent changes in lysosomal health in PBMC subsets. To explore this, the MFI of Lysotracker Red DND-99 (LTR), which accumulates in and stains sufficiently acidic lysosomes within cells, was quantified in monocytes and T cells after treatment with the stimulation paradigm. It was observed that lysosomal content, quantified from LTR MFI, was not significantly affected by PD status in monocytes, CD4+ T cells, or CD8+ T cells (FIG. 14A-E). However, CD8^{dim} T cells from RBD patients showed increased lysosomal content relative to all other groups in vehicle treated condition (FIG. 14F).

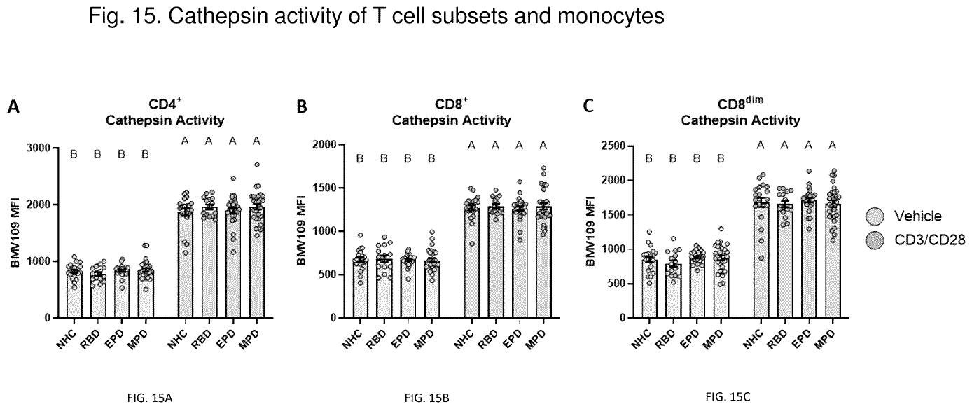

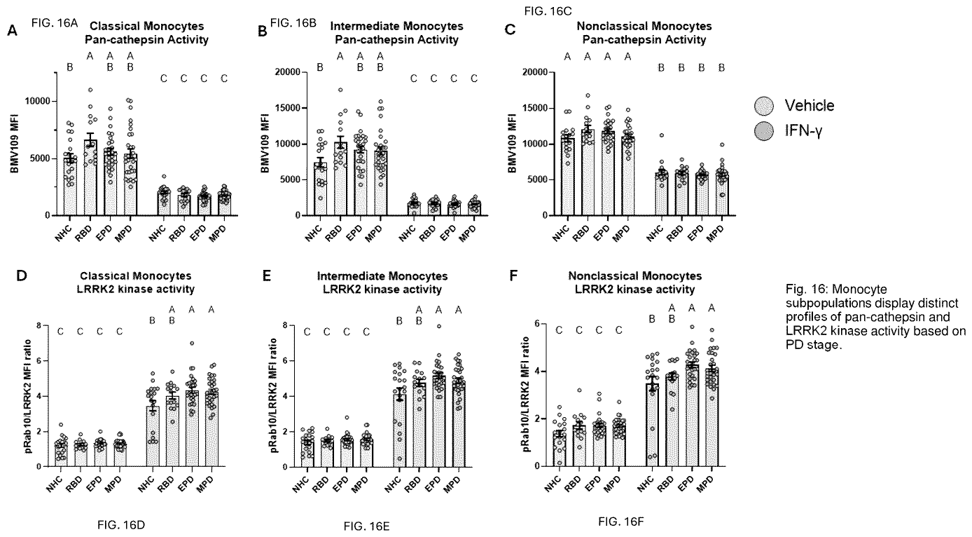

Next, the pan-cathepsin fluorescent probe BMV109 was used to quantify lysosomal cathepsin activity as a marker of lysosomal function in these cells. It was first observed that classical monocytes from RBD patients exhibited increased cathepsin activity relative to NHCs in vehicle condition (FIG. 16A). Additionally, intermediate monocytes from RBD patients showed increased cathepsin activity in vehicle condition relative to NHCs (FIG. 16B). No differences in cathepsin activity were observed across groups in nonclassical monocytes (FIG. 16C) or T cell populations (FIG. 15). Collectively, these data suggest that PD is associated with an upregulation of baseline lysosomal degradative capacity in specific monocyte subsets, and this upregulation can be detected in the prodromal stage of disease.

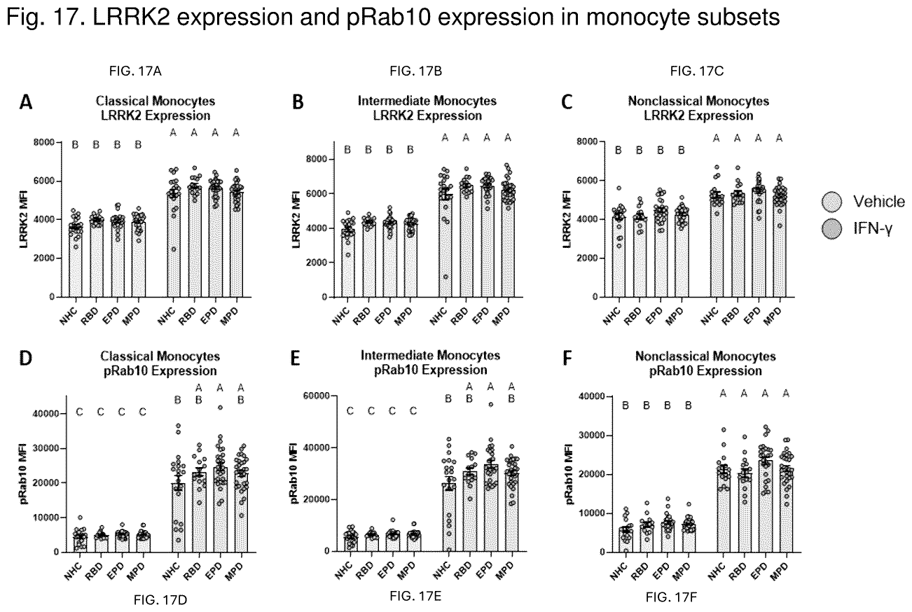

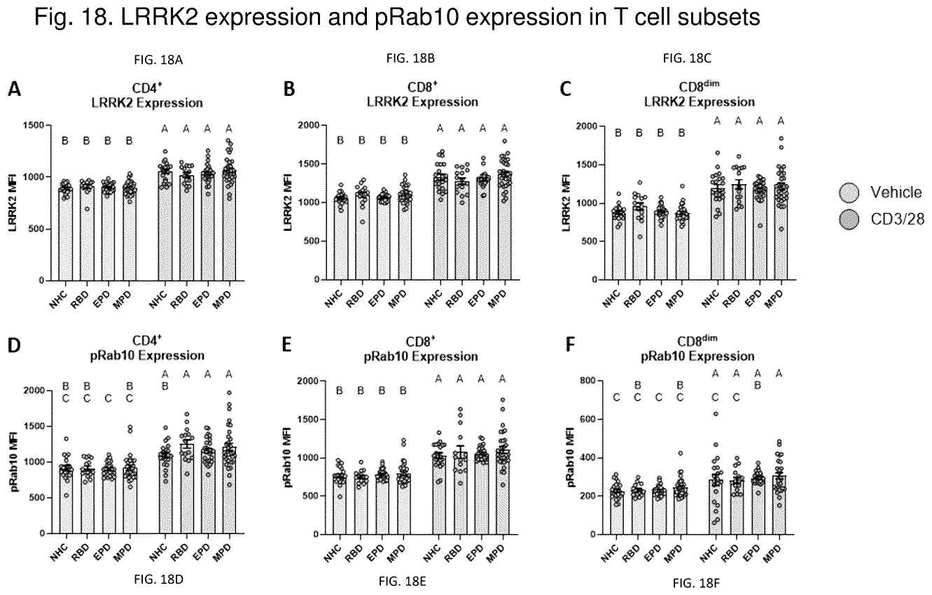

Alterations in leucine-rich repeat kinase 2 (LRRK2) expression have been heavily implicated in lysosomal biology and function^{51, 52}, and the gain-of-kinase mutation LRRK2-G2019S is one of the most common causes of genetic PD⁵³. Additionally, previous work in this lab described changes in the expression of both LRRK2 and phosphorylated Rab10 (pRab10), a LRRK2 substrate indicative of kinase activity, in peripheral immune cells from iPD patients³⁴. Recent observations have also defined a role for LRRK2 in suppressing lysosomal degradative activity and regulating cytokine secretion^{34, 54}. In light of this evidence, it was sought to explore the effects of PD progression on LRRK2 expression and kinase activity in stimulated PBMC subsets. It was found that IFNγ treatment increased both LRRK2 and pRab10 expression in all monocyte subtypes (FIG. 17). Relative LRRK2 kinase activity was assessed by normalizing pRab10 MFI to LRRK2 MFI, and this revealed significantly increased stimulation-dependent LRRK2 kinase activity in all monocyte subtypes from early PD and moderate PD groups relative to NHCs (FIG. 17D-F). Meanwhile, LRRK2 expression and kinase activity in T cell subsets were similar across different stages of PD (FIG. 18). Jointly, these experiments indicate that PD is associated with increases in stimulation-dependent LRRK2 activity, and these effects are cell-type specific.

### REFERENCES

1. Cheng, H. C., Ulane, C. M. & Burke, R. E. Clinical progression in Parkinson disease and the neurobiology of axons. Ann Neurol 67, 715-725 (2010).
2. Galbiati, A., Verga, L., Giora, E., Zucconi, M. & Ferini-Strambi, L. The risk of neurodegeneration in REM sleep behavior disorder: A systematic review and meta-analysis of longitudinal studies. Sleep Med Rev 43, 37-46 (2019).
3. Postuma, R. B. et al. Risk and predictors of dementia and parkinsonism in idiopathic REM sleep behaviour disorder: a multicentre study. Brain 142, 744-759 (2019).
4. St Louis, E. K. & Boeve, B. F. REM Sleep Behavior Disorder: Diagnosis, Clinical Implications, and Future Directions. Mayo Clin Proc 92, 1723-1736 (2017).
5. Tansey, M. G. et al. Inflammation and immune dysfunction in Parkinson disease. Nat Rev Immunol 22, 657-673 (2022).
6. Kustrimovic, N. et al. Parkinson's disease patients have a complex phenotypic and functional Th1 bias: cross-sectional studies of CD4+Th1/Th2/T17 and Treg in drug-naïve and drug-treated patients. J Neuroinflammation 15, 205 (2018).
7. Schlachetzki, J. C. M. et al. A monocyte gene expression signature in the early clinical course of Parkinson's disease. Sci Rep 8, 10757 (2018).
8. Muñoz-Delgado, L. et al. Peripheral inflammatory immune response differs among sporadic and familial Parkinson's disease. NPJ Parkinsons Dis 9, 12 (2023).
9. Qu, Y. et al. A systematic review and meta-analysis of inflammatory biomarkers in Parkinson's disease. NPJ Parkinsons Dis 9, 18 (2023).
10. Qin, X. Y., Zhang, S .P., Cao, C., Loh, Y. P. & Cheng, Y. Aberrations in Peripheral Inflammatory Cytokine Levels in Parkinson Disease: A Systematic Review and Meta-analysis. JAMA Neurol 73, 1316-1324 (2016).
11. Hull, A., Atilano, M. L., Gergi, L. & Kinghorn, K. J. Lysosomal storage, impaired autophagy and innate immunity in Gaucher and Parkinson's diseases: insights for drug discovery. Philos Trans R Soc Lond B Biol Sci 379, 20220381 (2024).
12. Dzamko, N., Rowe, D. B. & Halliday, G. M. Increased peripheral inflammation in asymptomatic leucine-rich repeat kinase 2 mutation carriers. Mov Disord 31, 889-897 (2016).
13. D'Esposito, V. et al. Lifestyle and Dietary Habits Affect Plasma Levels of Specific Cytokines in Healthy Subjects. Front Nutr 9, 913176 (2022).
14. Nilsonne, G., Lekander, M., Åkerstedt, T., Axelsson, J. & Ingre, M. Diurnal Variation of Circulating Interleukin-6 in Humans: A Meta-Analysis. PLoS One 11, e0165799 (2016).
15. Nguyen, T. H. O. et al. Immune cellular networks underlying recovery from influenza virus infection in acute hospitalized patients. Nat Commun 12, 2691 (2021).
16. Marchewka, W. M. et al. Sex-specific associations between the environmental exposures and low-grade inflammation and increased blood pressure in young, healthy subjects. Sci Rep 14, 9588 (2024).
17. Yamashita, K. Y., Bhoopatiraju, S., Silverglate, B. D. & Grossberg, G. T. Biomarkers in Parkinson's disease: A state of the art review. Biomarkers in Neuropsychiatry 9, 100074 (2023).
18. Tönges, L. et al. Blood-based biomarker in Parkinson's disease: potential for future applications in clinical research and practice. J Neural Transm (Vienna) 129, 1201-1217 (2022).
19. Zhang, H. et al. Plasma immune markers in an idiopathic REM sleep behavior disorder cohort. Parkinsonism Relat Disord 78, 145-150 (2020).
20. Kim, R. et al. Serum TNF-α and neurodegeneration in isolated REM sleep behavior disorder. Parkinsonism Relat Disord 81, 1-7 (2020).
21. Souquette, A. & Thomas, P. G. Variation in the basal immune state and implications for disease. Elife 13(2024 ).
22. De Picker, L. et al. Immune and Neuroendocrine Trait and State Markers in Psychotic Illness: Decreased Kynurenines Marking Psychotic Exacerbations. Front Immunol 10, 2971 (2019).
23. Cocoros, N.M. et al. Long-term Risk of Parkinson Disease Following Influenza and Other Infections. JAMA Neurol 78, 1461-1470 (2021).
24. Caggiu, E. et al. Homologous HSV1 and alpha-synuclein peptides stimulate a T cell response in Parkinson's disease. J Neuroimmunol 310, 26-31 (2017).
25. Hu, C. et al. Immune cell metabolism and metabolic reprogramming. Mol Biol Rep 49, 9783-9795 (2022).
26. Moon, H. E. & Paek, S. H. Mitochondrial Dysfunction in Parkinson's Disease. Exp Neurobiol 24, 103-116 (2015).
27. Dehay, B. et al. Lysosomal impairment in Parkinson's disease. Mov Disord 28, 725-732 (2013).
28. Wallings, R. L., Humble, S. W., Ward, M. E. & Wade-Martins, R. Lysosomal Dysfunction at the Centre of Parkinson's Disease and Frontotemporal Dementia/Amyotrophic Lateral Sclerosis. Trends Neurosci 42, 899-912 (2019).
29. Schirinzi, T. et al. Pattern of Mitochondrial Respiration in Peripheral Blood Cells of Patients with Parkinson&rsquo; s Disease. International Journal of Molecular Sciences 23, 10863 (2022).
30. Qadri, R. et al. Alterations in mitochondrial membrane potential in peripheral blood mononuclear cells in Parkinson's Disease: Potential for a novel biomarker. Restor Neurol Neurosci 36, 719-727 (2018).
31. Miki, Y. et al. Alteration of autophagy-related proteins in peripheral blood mononuclear cells of patients with Parkinson's disease. Neurobiol Aging 63, 33-43 (2018).
32. Smith, A .M. et al. Mitochondrial dysfunction and increased glycolysis in prodromal and early Parkinson's blood cells. Mov Disord 33, 1580-1590 (2018).
33. Hughes, L. P. et al. Glucocerebrosidase Activity is Reduced in Cryopreserved Parkinson's Disease Patient Monocytes and Inversely Correlates with Motor Severity. J Parkinsons Dis 11, 1157-1165 (2021).
34. Wallings, R. L. et al. WHOPPA Enables Parallel Assessment of Leucine-Rich Repeat Kinase 2 and Glucocerebrosidase Enzymatic Activity in Parkinson's Disease Monocytes. Front Cell Neurosci 16, 892899 (2022).
35. Tian, J. et al. Specific immune status in Parkinson's disease at different ages of onset. NPJ Parkinsons Dis 8, 5 (2022).
36. Ong, S. M. et al. The pro-inflammatory phenotype of the human non-classical monocyte subset is attributed to senescence. Cell Death Dis 9, 266 (2018).
37. Clutton, G. T., Weideman, A. M. K., Goonetilleke, N. P. & Maurer, T. An expanded population of CD8(dim) T cells with features of mitochondrial dysfunction and senescence is associated with persistent HIV-associated Kaposi's sarcoma under ART. Front Cell Dev Biol 10, 961021 (2022).
38. Falanga, Y. T. et al. High pathogen burden in childhood promotes the development of unconventional innate-like CD8+ T cells. JCI Insight 2(2017 ).
39. Xiao, Z., Mescher, M. F. & Jameson, S. C. Detuning CD8 T cells: down-regulation of CD8 expression, tetramer binding, and response during CTL activation. J Exp Med 204, 2667-2677 (2007).
40. Rodríguez-Rodríguez, N., Rosetti, F. & Crispín, J. C. CD8 is down(regulated) for tolerance. Trends Immunol 45, 442-453 (2024).
41. Zhang, L., Fung-Leung, W. & Miller, R. G. Down-regulation of CD8 on mature antigen-reactive T cells as a mechanism of peripheral tolerance. J Immunol 155, 3464-3471 (1995).
42. Schönrich, G. et al. Down-regulation of T cell receptors on self-reactive T cells as a novel mechanism for extrathymic tolerance induction. Cell 65, 293-304 (1991).
43. Ganeshan, K. & Chawla, A. Metabolic regulation of immune responses. Annu Rev Immunol 32, 609-634 (2014).
44. Wu, H. et al. Mitochondrial dysfunction promotes the transition of precursor to terminally exhausted T cells through HIF-1a-mediated glycolytic reprogramming. Nat Commun 14, 6858 (2023).
45. Richter, F. C., Saliutina, M., Hegazy, A. N. & Bergthaler, A. Take my breath away-mitochondrial dysfunction drives CD8(+) T cell exhaustion. Genes Immun 25, 4-6 (2024).
46. Bussi, C. et al. Lysosomal damage drives mitochondrial proteome remodelling and reprograms macrophage immunometabolism. Nat Commun 13, 7338 (2022).
47. Zorova, L. D. et al. Mitochondrial membrane potential. Anal Biochem 552, 50-59 (2018).
48. Scharping, N. E. et al. Mitochondrial stress induced by continuous stimulation under hypoxia rapidly drives T cell exhaustion. Nat Immunol 22, 205-215 (2021).
49. Papagiannakis, N. et al. Autophagy dysfunction in peripheral blood mononuclear cells of Parkinson's disease patients. Neurosci Lett 704, 112-115 (2019).
50. Jin, M. M. et al. A Critical Role of Autophagy in Regulating Microglia Polarization in Neurodegeneration. Front Aging Neurosci 10, 378 (2018).
51. Erb, M. L. & Moore, D. J. LRRK2 and the Endolysosomal System in Parkinson's Disease. J Parkinsons Dis 10, 1271-1291 (2020).
52. Madureira, M., Connor-Robson, N. & Wade-Martins, R. “LRRK2: Autophagy and Lysosomal Activity”. Front Neurosci 14, 498 (2020).
53. Bouhouche, A. et al. LRRK2 G2019S Mutation: Prevalence and Clinical Features in Moroccans with Parkinson's Disease. Parkinsons Dis 2017, 2412486 (2017).
54. Yadavalli, N. & Ferguson, S. M. LRRK2 suppresses lysosome degradative activity in macrophages and microglia through MiT-TFE transcription factor inhibition. Proc Natl Acad Sci U S A 120, e2303789120 (2023).
55. Peter, I. et al. Anti-Tumor Necrosis Factor Therapy and Incidence of Parkinson Disease Among Patients With Inflammatory Bowel Disease. JAMA Neurol 75, 939-946 (2018).
56. Gao, X., Chen, H., Schwarzschild, M. A. & Ascherio, A. Use of ibuprofen and risk of Parkinson disease. Neurology 76, 863-869 (2011).
57. Vijiaratnam, N. et al. Exenatide once weekly over 2 years as a potential disease-modifying treatment for Parkinson's disease: protocol for a multicentre, randomised, double blind, parallel group, placebo controlled, phase 3 trial: The ‘Exenatide-PD3’ study. BMJ Open 11, e047993 (2021).
58. Li, Y. et al. Efficacy of idebenone in the Treatment of iRBD into Synucleinopathies (EITRS): rationale, design, and methodology of a randomized, double-blind, multi-center clinical study. Front Neurol 13, 981249 (2022).
59. Zhao, Y., Shao, Q. & Peng, G. Exhaustion and senescence: two crucial dysfunctional states of T cells in the tumor microenvironment. Cell Mol Immunol 17, 27-35 (2020).
60. Ross, S. H. & Cantrell, D. A. Signaling and Function of Interleukin-2 in T Lymphocytes. Annu Rev Immunol 36, 411-433 (2018).
61. Thome, A. D. et al. Ex vivo expansion of dysfunctional regulatory T lymphocytes restores suppressive function in Parkinson's disease. NPJ Parkinsons Dis 7, 41 (2021).
62. Abou-Sleiman, P. M., Muqit, M. M. & Wood, N. W. Expanding insights of mitochondrial dysfunction in Parkinson's disease. Nat Rev Neurosci 7, 207-219 (2006).
63. Bose, A. & Beal, M. F. Mitochondrial dysfunction in Parkinson's disease. J Neurochem 139 Suppl 1, 216-231 (2016).
64. Park, J. S., Davis, R. L. & Sue, C. M. Mitochondrial Dysfunction in Parkinson's Disease: New Mechanistic Insights and Therapeutic Perspectives. Curr Neurol Neurosci Rep 18, 21 (2018).
65. Athauda, D. & Foltynie, T. Challenges in detecting disease modification in Parkinson's disease clinical trials. Parkinsonism Relat Disord 32, 1-11 (2016).
66. Simuni, T. et al. A biological definition of neuronal a-synuclein disease: towards an integrated staging system for research. Lancet Neurol 23, 178-190 (2024).
67. Höglinger, G.U. et al. A biological classification of Parkinson's disease: the SynNeurGe research diagnostic criteria. Lancet Neurol 23, 191-204 (2024).
68. Kang, J. H. et al. CSF biomarkers associated with disease heterogeneity in early Parkinson's disease: the Parkinson's Progression Markers Initiative study. Acta Neuropathol 131, 935-949 (2016).
69. Williams-Gray, C. H. et al. Serum immune markers and disease progression in an incident Parkinson's disease cohort (ICICLE-PD). Mov Disord 31, 995-1003 (2016).
70. Katare, P. B., Bagul, P. K., Dinda, A. K. & Banerjee, S. K. Toll-Like Receptor 4 Inhibition Improves Oxidative Stress and Mitochondrial Health in Isoproterenol-Induced Cardiac Hypertrophy in Rats. Front Immunol 8, 719 (2017).
71. Tresse, E. et al. Mitochondrial DNA damage triggers spread of Parkinson's disease-like pathology. Mol Psychiatry 28, 4902-4914 (2023).
72. Ongari, G. et al. Impaired Mitochondrial Respiration in REM-Sleep Behavior Disorder: A Biomarker of Parkinson's Disease? Mov Disord 39, 294-304 (2024).
73. Santos, D. et al. IL-1β Production by Intermediate Monocytes Is Associated with Immunopathology in Cutaneous Leishmaniasis. J Invest Dermatol 138, 1107-1115 (2018).
74. Dimitrov, S. et al. Differential TNF production by monocyte subsets under physical stress: blunted mobilization of proinflammatory monocytes in prehypertensive individuals. Brain Behav Immun 27, 101-108 (2013).
75. Pišlar, A., Tratnjek, L., Glavan, G., Živin, M. & Kos, J. Upregulation of Cysteine Protease Cathepsin X in the 6-Hydroxydopamine Model of Parkinson's Disease. Front Mol Neurosci 11, 412 (2018).
76. Yelamanchili, S. V., Chaudhuri, A. D., Flynn, C. T. & Fox, H. S. Upregulation of cathepsin D in the caudate nucleus of primates with experimental parkinsonism. Mol Neurodegener 6, 52 (2011).
77. Kim, K. R. et al. S-Nitrosylation of cathepsin B affects autophagic flux and accumulation of protein aggregates in neurodegenerative disorders. Cell Death Differ 29, 2137-2150 (2022).
78. Haeussler, S. et al. Autophagy compensates for defects in mitochondrial dynamics. PLoS Genet 16, e1008638 (2020).
79. Graef, M. & Nunnari, J. Mitochondria regulate autophagy by conserved signalling pathways. Embo j 30, 2101-2114 (2011).
80. Mutti, V. et al. LRRK2 Kinase Inhibition Attenuates Neuroinflammation and Cytotoxicity in Animal Models of Alzheimer's and Parkinson's Disease-Related Neuroinflammation. Cells 12(2023 ).
81. Wallings, R. L. et al. ASO-mediated knockdown or kinase inhibition of G2019S-Lrrk2 modulates lysosomal tubule-associated antigen presentation in macrophages. Mol Ther Nucleic Acids 34, 102064 (2023).

What is claimed is:

1. A method to diagnose if a subject is at risk of developing Parkinson's disease (PD), the method comprising steps of:
i. Obtaining immune cells from a subject; and
ii. Using the immune cells, conducting one or more of the following:
a. measuring ex vivo stimulation-evoked changes in cytokine secretion of immune cells from the subject;
b. measuring ex vivo stimulation-evoked changes in monocyte- or T cell subtype population frequencies of immune cells from the subject;
c. measuring ex vivo stimulation-evoked changes in the quantity of the mitochondria with negative membrane potential (healthy mitochondria) and total mitochondria, to determine the ratio of the healthy mitochondria;
d. measuring ex vivo stimulation-evoked changes in lysosomal function, which is assayed by measuring lysosomal cathepsin activity, and total lysosomal quantity, to determine the ratio of the functional lysosome; and/or
e. measuring changes in LRRK2 activity and LRRK2 protein level.

2. The method of claim 1, wherein the method comprises
i. Obtaining blood from the subject,
ii. isolating peripheral blood mononuclear cells (PBMCs) from the blood;
iii. isolating monocytes from the PBMC, and optionally further isolating subtypes of monocytes, wherein the subtypes comprise classical monocytes (CD14+/CD16−), intermediate monocytes (CD14+/CD16+), and non-classical (CD14dim/CD16+) monocytes;
iv. plating a certain number of monocytes or subtypes, and treating them with vehicle or human IFNγ;
v. isolating CD3⁺ T cells from the PBMC, and optionally further isolating subtypes of T cells, wherein the subtypes comprise CD4+ T cells, CD8+ T cells, and CD8dim T cells;
vi. plating a certain number of CD3⁺ T cells or subtypes, and treating them with vehicle or CD3/CD28, optionally CD3/CD28 coated on magnetic beads.

3. The method of claim 1, wherein ex vivo stimulation-evoked changes in cytokine secretion can be measured by steps including:
i. harvesting the monocyte-and T cell samples after ex vivo stimulation, respectively, and centrifuging them to obtain supernatant of each sample, which is conditioned media from cultured monocytes or T cells;
ii. measuring at least one cytokine selected from TNF, IL-1β, IL-2, IL-6, IL-8, and IL-10 in the supernatant with cytokine antibody/antibodies and secondary antibodies conjugated with electrochemiluminescent or fluorescent labels;
iii. normalizing the quantity of cytokines released from CD3/CD28 treated T cells to that released from vehicle treated T cells; and/or the quantity of cytokines released from IFNγ treated monocytes to that released from vehicle treated monocytes; and
vii. determining PD if ex vivo stimulation-evoked cytokine secretion of TNF, IL-1β, and IL-8 from monocytes has significantly increased; and determining moderate PD if ex vivo stimulation-evoked cytokine secretion of IL-2 and IL-10 from T cells has significantly reduced.

4. The method of claim 1, wherein ex vivo stimulation-evoked changes in monocyte- or T cell subtype population frequencies is measured by steps including:
i. incubating monocytes with electrochemiluminescence or fluorescence conjugated antibodies against cell surface antigens, wherein the cell surface antigens of monocytes are CD14 and/or CD16;
ii. incubating T cells with electrochemiluminescence or fluorescence conjugated antibodies against cell surface antigens, wherein the cell surface antigens of T cells are CD4 and/or CD8;
iii. performing flow cytometry;
iv. normalizing the quantity of each subtype of CD3/CD28 treated T cells to that of vehicle treated T cells; and/or the quantity of each subtype of IFNγ treated monocytes to that of vehicle treated monocytes; and
v. determine PD if flow cytometry analysis reveals that normalized frequency of stimulated classical monocytes has increased and that of intermediate- and nonclassical monocytes has decreased; and normalized frequency of stimulated CD8⁺ T cells has decreased, and that of CD8^{dim} T cells has increased.

5. The method of claim 1, wherein ex vivo stimulation-evoked changes in the quantity of the mitochondria with negative membrane potential (healthy mitochondria) and total mitochondria, to determine the ratio of the healthy mitochondria; and/or lysosomal cathepsin activity can be measured by steps including:
i. harvesting the monocyte-and T cell samples after ex vivo stimulation, respectively, and centrifuging them to obtain cell pellets of each sample, resuspending the cells in washing buffer, plating the cells, and centrifuging them;
ii. resuspending the cells in complete growth media containing a fluorescent agent for staining the mitochondria and a fluorescent agent for staining the mitochondria with negative membrane potential, and/or a fluorescent agent for staining pan cathepsin, and incubating the sample, optionally for about 1 hour at about 37° C. in the dark;
iii. after centrifugation, washing, and centrifugation, resuspending the cells in PBS containing a fluorescent agent indicating cell viability, and incubating the cells, optionally at room temperature for about 30 min in the dark;
iv. after centrifugation, washing, and centrifugation, resuspending the cells in PBS containing at least one of electrochemiluminescence or fluorescence conjugated T cell specific antibodies listed in Table 2 or electrochemiluminescence or fluorescence conjugated monocyte specific antibodies listed in Table 3, and incubating the cells, optionally at about 4° C. for about 20 min in the dark;
v. after centrifugation, washing, centrifugation, and resuspension in FACS buffer, performing live cell flow cytometry;
vi. calculating the ratio of the mitochondria with negative membrane potential to total mitochondria, and/or deciding lysosomal cathepsin activity;
vii. normalizing the mitochondrial ratio and/or cathepsin activity in CD3/CD28 treated T cells to those in vehicle treated T cells; and/or the mitochondrial ratio and/or cathepsin activity in IFNγ treated monocytes to those in vehicle treated monocytes; and
viii. determining moderate PD if CD8+ and CD8^{dim} T cells and nonclassical monocytes display reduced ratio after stimulation;
wherein, mitochondria staining and cathepsin staining of step ii is optionally performed in separate cells.

6. The method of claim 1, wherein the quantity of LRRK2, LRRK2 activity (i.e., pT73 Rab10), and/or the quantity of lysosome can be measured by steps including:
i. harvesting the monocyte-and T cell samples after ex vivo stimulation, respectively, and centrifuging them to obtain cell pellets of each sample, resuspending the cells in washing buffer, plating the cells, and centrifuging them;
ii. resuspending the cells in complete growth media containing a fluorescent agent for staining acidic lysosome, and incubating the sample, optionally for about 1 hour at about 37° C. in the dark;
iii. after centrifugation, washing, and centrifugation, resuspending the cells, optionally in PBS containing a fluorescent agent indicating cell viability, and incubating the cells, optionally at room temperature for about 30 min in the dark;
iv. after centrifugation, washing, and centrifugation, resuspending the cells, optionally in PBS containing about 1% paraformaldehyde (PFA), and incubating the cells, optionally at about 4° C. for about 30 min in the dark;
v. after centrifugation, washing, and centrifugation, resuspending the cells in permeabilization buffer, and incubating the cells on ice, optionally for about 15 min;
vi. adding anti-pT73 Rab10 antibody to the samples and incubating them, optionally at room temperature for about 30 min in the dark;
vii. after centrifugation, washing, and centrifugation, resuspending the cells in PBS containing electrochemiluminescence or fluorescence conjugated secondary antibodies against anti-pT73 Rab10 antibody, and incubating the samples, optionally at room temperature for about 30 min in the dark;
viii. after centrifugation, washing, and centrifugation, resuspending the cells in PBS containing electrochemiluminescence or fluorescence conjugated anti-LRRK2 antibody, and incubating the sample, optionally at about 4° C. for about 20 min in the dark;
ix. after centrifugation, washing, centrifugation, and resuspension in FACS buffer, performing fixed cell flow cytometry;
x. normalizing the quantity of LRRK2, LRRK2 activity, and/or the quantity of lysosome in CD3/CD28 treated T cells to those in vehicle treated T cells; and/or the quantity of LRRK2, LRRK2 activity, and/or the quantity of lysosome in IFNγ treated monocytes to those in vehicle treated monocytes; and
xi. determining PD if relative LRRK2 kinase activity assessed by normalizing pRab10 to LRRK2 has significantly increased after stimulation in all monocyte subtypes;
wherein, optionally lysosome staining of step iv can be performed separately.

7. A kit to diagnose if a subject is at risk of developing Parkinson's disease (PD), comprising:
i. at least one antibody detecting at least one of cytokines selected from TNF, IL-1β, IL-2, IL-6, IL-8, and IL-10, and antibodies conjugated with electrochemiluminescence or fluorescence labels (secondary antibodies) against cytokine antibody;
ii. at least one electrochemiluminescence or fluorescence conjugated antibody detecting T cells, selected from Table 2;
iii. at least one electrochemiluminescence or fluorescence conjugated antibody detecting monocytes, selected from Table 3;
iv. CD3/CD28 coated magnetic beads and human IFNγ;
v. fluorescent agent staining the mitochondria and fluorescent agent staining the mitochondria with negative membrane potential;
vi. fluorescent agent staining the lysosome and fluorescent agent indicating cathepsin activity;
vii. leucine-rich repeat kinase 2 (LRRK2) antibody with or without electrochemiluminescence or fluorescence label, pT73 Rab10 antibody with or without electrochemiluminescence or fluorescence label, and electrochemiluminescence or fluorescence conjugated antibodies (secondary antibodies) against LRRK2 antibody or pT73 Rab10 antibody; and
viii. PBS, MACS buffer, FACS buffer.

8. A method of treating a patient at risk of developing Parkinson's disease (PD), the method comprising administering a PD therapy, wherein the patient at risk exhibits increased ex vivo IFNγ stimulation-evoked cytokine secretion of TNF, IL-1γ, and IL-8 from monocytes, and at least one of the following:
i. increase or decrease of monocyte subtype_or T cell subtype_when monocytes are ex vivo IFNγ stimulated or T cells are ex vivo CD3/CD28 stimulated;
ii. decrease of ratio of mitochondria with negative membrane potential to total mitochondria in CD8+T cells when monocytes are T cells are ex vivo CD3/CD28 stimulated;
increase or decrease of lysosome quantity and/or lysosome cathepsin activity in monocytes when monocytes are ex vivo IFNγ stimulated or CD8+ T cells when monocytes are T cells are ex vivo CD3/CD28 stimulated; or
iv. increase of LRRK2 activity in monocytes when monocytes are ex vivo IFNγ stimulated.
